SEQUENCE LISTING (1 sequence)

SEQ ID NO: 1 - protein, 14 aa, Artificial
  Synthetic polypeptide
IEGPTLRQWLAARA

(19) United States
(12) Patent Application Publication
(10) Pub. No.: US 2009/0088371 A1
(43) Pub. Date: Apr. 2, 2009

(54) COMBINATION THERAPY WITH SYK KINASE INHIBITOR

(75) Inventor: Elliott Grossbard, San Francisco, CA (US)

Correspondence Address: RIGEL PHARMACEUTICALS INC., C/O FOLEY & LARDNER LLP, 975 PAGE MILL ROAD, PALO ALTO, CA, 94304, US

(73) Assignee: Rigel Pharmaceuticals, Inc.

(21) Appl. No.: 12/199,766

(22) Filed: Aug. 27, 2008

Related U.S. Application Data
(60) Provisional application No. 60/968,487, filed on Aug. 28, 2007.

(51) Int. Cl. A61K 38/14; A61K 31/675; A61K 38/10; A61P 7/00; A61K 31/415; A61K 38/02

(52) U.S. Cl. 514/8; 514/81; 514/2; 514/14; 514/407

(57) ABSTRACT

The invention encompasses methods of increasing platelet levels in a patient having or at risk for immune thrombocytopenia comprising co-administering a Syk kinase inhibitor and a thrombopoietin receptor agonist, and methods of treating thrombocytopenia comprising co-administering a Syk kinase inhibitor and a thrombopoietin receptor agonist to a patient in need thereof, as well as pharmaceutical compositions for use in these methods.

### A. CROSS-REFERENCE TO RELATED APPLICATIONS

This application claims the benefit of U.S. provisional patent application No. 60/968,487 filed 28 Aug. 2007, which is incorporated herein by reference in its entirety.

### B. FIELD

The present invention relates to improved methods of treating and or preventing immune thrombocytopenia, particularly immune thrombocytopenia purpura.

### C. BACKGROUND

Thrombocytopenia is characterized by low platelet numbers in the blood, commonly leading to bleeding disorders in which the blood fails to clot properly. Because platelets (thrombocytes) are necessary for blood clotting, when their numbers are very low, a patient can be at risk of death from catastrophic hemorrhage.

Thrombocytopenia, or a predisposition toward developing thrombocytopenia, may be inherited or acquired. Acquired thrombocytopenia may be caused by, inter alia, immune causes, such as immune thrombocytopenic purpura (ITP), neonatal alloimmune thrombocytopenia or post-transfusion purpura (PTP), and non immune causes, including disseminated intravascular coagulopathy (DIC), thrombotic thromobcytopenic purpura (TTP) heparin-induced thrombocytopenia (HIT), splenomegaly/splenic sequestration, turbulent blood flow, drug-induced marrow suppression, viral infection, bacterial infection, alcoholism/bone marrow suppression, myelodysplastic syndrome (MDS), anaplastic anemia, hematologic malignancy and solid tumor infiltration of bone marrow. See, Drachman, Blood 103(2):390-398 (2004).

Treatment of thrombocytopenia can vary depending on its cause and severity. Typically, in acquired thrombocytopenia, identification and correction of the condition causing the thrombocytopenia, e.g. ceasing administration of heparin in heparin-induced thrombocytopenia, is the preferred course of treatment. However, in many situations, the cause cannot be identified or cannot be eliminated. In these cases, a common treatment is platelet transfusion. However, such transfusions may lose their effectiveness with repeated use due to development of platelet alloantibodies.

Adult immune thrombocytopenia purpura (also known as idiopathic thrombocytopenia purpura or immunologic thrombocytopenia purpura) typically results from development of an antibody directed against a structural platelet antigen. First line treatment for ITP typically begins with an oral corticosteroid, which is then tapered off. However, most patients fail to respond adequately, or relapse as the corticosteroid is tapered. 50-60% of patients can achieve remission with a splenectomy. The efficacy of other drugs for patients refractory to corticosteroids and/or splenectomy is unproven. Accordingly, new medications and regimens for treating and/or preventing immune thrombocytopenia purpura are needed.

### SUMMARY OF THE INVENTION

This invention is directed to methods of increasing platelet levels in a patient having or at risk for immune thrombocytopenia comprising co-administering a Syk kinase inhibitor and a thrombopoietin receptor agonist. This invention also provides methods of treating thrombocytopenia comprising co-administering a Syk kinase inhibitor and a thrombopoietin receptor agonist to a patient in need thereof.

In one implementation, the Syk kinase inhibitor is a 2,4-pyrimidinediamine compound of formula I:

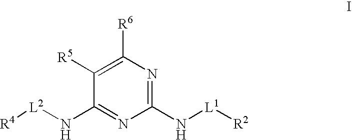

a salt, hydrate, solvate, N-oxide or prodrug thereof, wherein:
L¹ and L², independently of each other, are selected from the group consisting of a direct bond, a (C1-C3) alkylene optionally substituted with one or more of the same or different R⁹ groups, and 1-3 membered heteroalkyldiyl optionally substituted with one or more of the same or different R⁹ groups;
R² is selected from the group consisting of (C1-C6) alkyl optionally substituted with one or more of the same or different R⁸ groups, (C2-C6) alkenyl optionally substituted with one or more of the same or different R⁸ groups, (C2-C6) alkynyl optionally substituted with one or more of the same or different R⁸ groups, (C3-C8) cycloalkyl optionally substituted with one or more of the same or different R⁸ groups, 3-8 membered heterocyclyl optionally substituted with one or more of the same or different R⁸ groups, (C5-C15) aryl optionally substituted with one or more of the same or different R⁸ groups, and 5-15 membered heteroaryl optionally substituted with one or more of the same or different R⁸ groups;
R⁴ is selected from the group consisting of hydrogen, (C1-C6) alkyl optionally substituted with one or more of the same or different R⁸ groups, (C2-C6) alkenyl optionally substituted with one or more of the same or different R⁸ groups, (C2-C6) alkynyl optionally substituted with one or more of the same or different R⁸ groups, (C3-C8) cycloalkyl optionally substituted with one or more of the same or different R⁸ groups, 3-8 membered heterocyclyl optionally substituted with one or more of the same or different R⁸ groups, (C5-C15) aryl optionally substituted with one or more of the same or different R⁸ groups, and 5-15 membered heteroaryl optionally substituted with one or more of the same or different R⁸ groups;
R⁵ is selected from the group consisting of R⁶, (C2-C6) alkenyl optionally substituted with one or more of the same or different R⁸ groups, (C2-C6) alkynyl optionally substituted with one or more of the same or different R⁸ groups, and (C3-C8) cycloalkyl optionally substituted with one or more of the same or different R⁸ groups;
each R⁶, independently of the others, is selected from the group consisting of hydrogen, —OR^{d}, —SR^{d}, (C1-C3) haloalkyloxy, (C1-C3) perhaloalkyloxy, —NR^{c}R^{c}, halogen, (C1-C3) haloalkyl, (C1-C3) perhaloalkyl, —CN, —NC, —OCN, —SCN, —NO, —NO₂, —N₃, —S(O)R^{d}, —S(O)₂R^{d}, —S(O)NR^{c}R^{c}, —S(O)₂NR^{c}R^{c}, —OS(O)₂R^{d}, —OS(O)₂R^{d}, —OS(O)₂OR^{d}, —OS(O)NR^{c}R^{c}, —OS(O)₂NR^{c}R^{c}, —C(O)R^{d}, —C(O)OR^{d}, —C(O)NR^{c}R^{c}, —C(NH)NR^{c}R^{c}, —OC(O)R^{d}, —SC(O)R^{d}, —OC(O)OR^{d}, —SC(O)OR^{d}, —OC(O)NR^{c}R^{c}, —SC(O)NR^{c}R^{c}, —OC(NH)NR^{c}R^{c}, —SC(NH)NR^{c}R^{c}, —[NHC(O)]ₙR^{d}, —[NHC(O)]ₙOR^{d}, R[NHC(O)]ₙR^{c}R^{c}, R[NHC(NH)]ₙR^{c}R^{c}, (C5-C10) aryl optionally substituted with one or more of the same or different R⁸ groups, (C6-C16) arylalkyl optionally substituted with one or more of the same or different R⁸ groups, 5-10 membered heteroaryl optionally substituted with one or more of the same or different R⁸ groups, and 6-16 membered heteroarylalkyl optionally substituted with one or more of the same or different R⁸ groups;
R⁸ is selected from the group consisting of R^{e}, R^{b}, R^{e} substituted with one or more of the same or different R^{a} or R^{b}, —OR^{a} substituted with one or more of the same or different R^{a} or R^{b}, —B(OR^{a})₂, —B(NR^{c}R^{c})₂, —(CH₂)ₘ—R^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂, —O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b}, —C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b}, —NH—(CHR^{a})ₘ—R^{b}, —N[(CH₂)ₘR^{b}]₂, —NH—C(O)—NH—(CH₂)ₘ—R^{b}, —NH—C(O)—(CH₂)ₘ—CHR^{b}R^{b} and —NH—(CH₂)ₘ—C(O)—NH—(CH₂)ₘ—R^{b};
each R⁹, independently of the others, is selected from the group consisting of (C1-C6) alkyl, —OR^{a}, —C(O)OR^{a}, (C5-C10) aryl optionally substituted with one or more of the same or different halogens, phenyl optionally substituted with one or more of the same or different halogens, and 5-10 membered heteroaryl optionally substituted with one or more of the same or different halogens;
each R^{a}, independently of the others, is selected from the group consisting of hydrogen, (C1-C6) alkyl, (C3-C8) cycloalkyl, (C4-C11) cycloalkylalkyl, (C5-C10) aryl, (C6-C16) arylalkyl, 2-6 membered heteroalkyl, 3-8 membered heterocyclyl, 4-11 membered heterocyclylalkyl, 5-10 membered heteroaryl and 6-16 membered heteroarylalkyl;
each R^{b}, independently of the others, is a suitable group selected from the group consisting of ═O, —OR^{d}, (C1-C3) haloalkyloxy, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{c}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c}, independently of the others, is a progroup or R^{a}, or, alternatively, two R^{c} are taken together with the nitrogen atom to which they are bonded to form a 5 to 8-membered heterocyclyl or heteroaryl which may optionally include one or more of the same or different additional heteroatoms and which may optionally be substituted with one or more of the same or different R^{a} or suitable R^{b} groups;
each R^{d}, independently of the others, is a progroup or R^{a};
each R^{e}, independently of the others, is selected from the group consisting of (C1-C6) alkyl, (C3-C8) cycloalkyl, (C4-C11) cycloalkylalkyl, (C5-C10) aryl, (C6-C16) arylalkyl, 2-6 membered heteroalkyl, 3-8 membered heterocyclyl, 4-11 membered cycloheteroalkylalkyl, 5-10 membered heteroaryl and 6-16 membered heteroarylalkyl;
each m, independently of the others, is an integer from 1 to 3; and
each n, independently of the others, is an integer from 0 to 3.

In another implementation, the Syk kinase inhibitor is a 2,4-pyrimidinediamine compound of formula II:

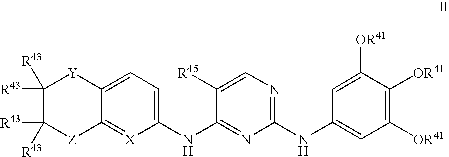

or a pharmaceutically acceptable salt or N-oxide thereof, wherein
X is selected from the group consisting of N and CH;
Y is selected from the group consisting of O, S, SO, SO₂, SONR⁴¹, NH, and NR⁴²;
Z is selected from the group consisting of O, S, SO, SO₂, SONR⁴¹, NH, and NR⁴²;
each R⁴¹, independently of the others, is hydrogen or lower alkyl;
each R⁴², independently of the others, is selected from the group consisting of alkyl, substituted alkyl, alkenyl, substituted alkenyl, alkynyl, substituted alkynyl, cycloalkynyl, substituted cycloalkynyl, aryl, substituted aryl, cycloalkyl, substituted cycloalkyl, heteroaryl, substituted heteroaryl, heterocyclyl, substituted heterocyclyl, hydroxyl, alkoxy, substituted alkoxy, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxyl ester, (carboxyl ester)oxy, and —(CRR)ₙ—R^{p}, wherein
   each R, independently of the others, is selected from the group consisting of hydrogen, lower alkyl and halo;
   n is an integer from 0 to 4; and
   R^{p} is selected from the group consisting of phosphate, phosphate ester, phosphonate, phosphorodiamidate, phosphoramidate monoester, phosphoramidate diester, cyclic phosphoramidate, cyclic phosphorodiamidate, phosphonamidate, and cyclic phosphonamidate;
each R⁴³, independently of the others, is selected from the group consisting of hydrogen, alkyl, substituted alkyl, alkoxy, substituted alkoxy, alkenyl, substituted alkenyl, alkynyl, substituted alkynyl, cycloalkynyl, substituted cycloalkynyl, alkynyloxy, amino, substituted amino, aryl, substituted aryl, aryloxy, substituted aryloxy, cyano, cycloalkyl, substituted cycloalkyl, cycloalkoxy, substituted cycloalkoxy, heteroaryl, substituted heteroaryl, heteroaryloxy, substituted heteroaryloxy, heterocyclyl, substituted heterocyclyl, heterocyclyloxy, substituted heterocyclyloxy, aminocarbonyl, aminocarbonyloxy, carboxyl, carboxyl ester, (carboxyl ester)oxy, nitro, and halo, or, alternatively, two R⁴³ bonded to the same carbon atom are taken together to form an oxo (═O), ═NH or ═NR⁴⁴ group and the other two R⁴³ are as defined above;
each R⁴⁴, independently of the others, is selected from the group consisting of (C1-C6) alkyl and (C5-C14) aryl; and
R⁴⁵ is selected from the group consisting of alkyl, substituted alkyl, alkoxy, substituted alkoxy, amino, substituted amino, carboxyl, carboxyl ester, cyano, halo, nitro, alkenyl, substituted alkenyl, alkynyl and substituted alkynyl.

### BRIEF DESCRIPTION OF THE DRAWINGS

FIG. 1 shows the effect of Compound 1 treatment on platelet counts in C57BL/6 mice.

FIG. 2 shows the effect of Compound 1 on antibody-mediated thrombocytopenia.

### DETAILED DESCRIPTION

### A. Definitions

As used herein, the following definitions shall apply unless otherwise indicated.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂—)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 hydrogens replaced with substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, alkynyl, substituted alkynyl, cycloalkynyl, substituted cycloalkynyl guanidino, substituted guanidino, halo, hydroxy, heteroaryl, substituted heteroaryl, heteroaryloxy, substituted heteroaryloxy, heteroarylthio, substituted heteroarylthio, heterocyclyl, substituted heterocyclyl, heterocyclyloxy, substituted heterocyclyloxy, heterocyclylthio, substituted heterocyclylthio, nitro, SO₃H, sulfonyl, sulfonyloxy, thioacyl, thiol, alkylthio, and substituted alkylthio, wherein said substituents are defined herein. In some embodiments, the alkyl has 1 to 3 of the aforementioned groups. In other embodiments, the alkyl has 1 to 2 of the aforementioned groups.

“Heteroalkyl” refers to an alkyl group in which one or more of the carbon atoms are each independently replaced with the same or different heteroatoms or heteroatomic groups. Typical heteroatoms and/or heteroatomic groups which can replace the carbon atoms include —O—, —S—, —NR—, —S(O)—, —P(H)—, —S(O)₂—, and the like.

“Alkylene” refers to divalent saturated aliphatic hydrocarbyl groups preferably having from 1 to 6 and more preferably 1 to 3 carbon atoms that are either straight-chained or branched. This term is exemplified by groups such as methylene (—CH₂—), ethylene (—CH₂CH₂—), n-propylene (—CH₂CH₂CH₂—), iso-propylene (—CH₂CH(CH₃)—) or (—CH(CH₃)CH₂—), and the like.

“Heteroalkyldivl” refers to an alkylene group in which one or more of the carbon atoms are each independently replaced with the same or different heteroatoms or heteroatomic groups. Typical heteroatoms and/or heteroatomic groups which can replace the carbon atoms include —O—, —S—, —NR—, —S(O)—, —P(H)—, —S(O)₂—, and the like.

“Substituted alkylene” refers to an alkylene group having from 1 to 3 hydrogens replaced with substituents selected from the group consisting of alkyl, substituted alkyl, alkoxy, substituted alkoxy, acyl, acylamino, acyloxy, amino, substituted amino, aminocarbonyl, aryl, substituted aryl, aryloxy, substituted aryloxy, cyano, halogen, hydroxyl, nitro, carboxyl, carboxyl ester, cycloalkyl, substituted cycloalkyl, heteroaryl, substituted heteroaryl, heterocyclyl, substituted heterocyclyl, and oxo wherein said substituents are defined herein. In some embodiments, the alkylene has 1 to 2 of the aforementioned groups. It is to be noted that when the alkylene is substituted by an oxo group, 2 hydrogens attached to the same carbon of the alkylene group are replaced by “═O”.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like.

“Substituted alkoxy” refers to the group —O-(substituted alkyl), wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)— cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclyl, and —NR²⁰C(O)substituted heterocyclyl, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclyl-C(O)O—, and substituted heterocyclyl-C(O)O—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Amino” refers to the group —NH₂.

“Substituted amino” refers to the group —NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, substituted heterocyclyl, where one of R²¹ and R²² is sulfonyl, and wherein R²¹ and R²² are optionally joined together with the nitrogen bound thereto to form a heterocyclic or substituted heterocyclic group, provided that R²¹ and R²² are not both hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, sulfonyl, substituted heteroaryl, heterocyclyl, and substituted heterocyclyl are as defined herein. When R²¹ is hydrogen and R²² is alkyl, the substituted amino group is sometimes referred to herein as “alkylamino.” When R²¹ and R²² are alkyl, the substituted amino group is sometimes referred to herein as “dialkylamino.” When referring to a monosubstituted amino, it is meant that either R²¹ or R²² is hydrogen, but not both. When referring to a disubstituted amino, it is meant that neither R²¹ nor R²² is hydrogen.

“Aminocarbonyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Aminothiocarbonyl” refers to the group —C(S)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Aminocarbonylamino” refers to the group —NR²⁰C(O)NR²¹R²², wherein R²⁰ is hydrogen or alkyl and R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl and substituted heterocyclyl are as defined herein.

“Aminothiocarbonylamino” refers to the group —NR²⁰C(S)NR²¹R²², wherein R²⁰ is hydrogen or alkyl and R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl and substituted heterocyclyl are as defined herein.

“Aminocarbonyloxy” refers to the group —O—C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl and substituted heterocyclyl are as defined herein.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, substituted heterocyclyl and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl and substituted heterocyclyl are as defined herein.

“Aminosulfonyloxy” refers to the group —O—SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl; R²¹ and R²² are optionally joined together with the nitrogen bound thereto to form a heterocyclic or substituted heterocyclic group; and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Aminosulfonylamino” refers to the group —NR²⁰—SO₂NR²¹R²², wherein R²⁰ is hydrogen or alkyl and R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl and substituted heterocyclyl are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Amidino” refers to the group —C(═NR³⁰)NR³¹R³², wherein R³¹ and R³² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R³¹ and R³² are optionally joined together with the nitrogen bound thereto to form a heterocyclic or substituted heterocyclic group. R³⁰ is selected from the group consisting of hydrogen, alkyl, substituted alkyl, alkenyl, substituted alkenyl, alkynyl, substituted alkynyl, cycloalkynyl, substituted cycloalkynyl, aryl, substituted aryl, cycloalkyl, substituted cycloalkyl, cycloalkenyl, substituted cycloalkenyl, heteroaryl, substituted heteroaryl, heterocyclyl, substituted heterocyclyl, nitro, nitroso, hydroxy, alkoxy, cyano, —N═N—N-alkyl, —N═N—N-substituted alkyl, —N(alkyl)SO₂-alkyl, —N(alkyl)SO₂-substituted alkyl, —N═N═N-alkyl, —N═N═N-substituted alkyl, acyl, —SO₂-alkyl and —SO₂-substituted alkyl, wherein alkyl, substituted alkyl, alkenyl, substituted alkenyl, alkynyl, substituted alkynyl, cycloalkynyl, substituted cycloalkynyl, cycloalkyl, substituted cycloalkyl, cycloalkenyl, substituted cycloalkenyl, aryl, substituted aryl, heteroaryl, substituted heteroaryl, heterocyclyl, substituted heterocyclyl, nitro, nitroso, hydroxy, alkoxy, and cyano are as defined herein. One of R³¹ and R³² along with R³⁰ are optionally joined together with the nitrogens bound thereto and the intervening carbon of the amidine group to form a cyclic amidine.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like), provided that the point of attachment is through an atom of the aromatic aryl group. Preferred aryl groups include phenyl and naphthyl.

“Substituted arvl” refers to aryl groups having 1 to 5 hydrogens replaced with substituents independently selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclyl, substituted heterocyclyl, heterocyclyloxy, substituted heterocyclyloxy, heterocyclylthio, substituted heterocyclylthio, nitro, SO₃H, sulfonyl, sulfonyloxy, thioacyl, thiol, alkylthio, and substituted alkylthio, wherein said substituents are as defined herein. In some embodiments, the aryl has 1 to 3 of the aforementioned groups. In other embodiments, the aryl has 1 to 2 of the aforementioned groups.

“Aryloxy” refers to the group —O-aryl, wherein aryl is as defined herein, including, by way of example, phenoxy, naphthoxy, and the like.

“Substituted arvloxy” refers to the group —O-(substituted aryl), wherein substituted aryl is as defined herein.

“Arylthio” refers to the group —S-aryl, wherein aryl is as defined herein. In other embodiments, sulfur may be oxidized to —S(O)— or —SO₂— moieties. The sulfoxide may exist as one or more stereoisomers.

“Substituted arvlthio” refers to the group —S-(substituted aryl), wherein substituted aryl is as defined herein. In other embodiments, sulfur may be oxidized to —S(O)— or —SO₂— moieties. The sulfoxide may exist as one or more stereoisomers.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. Such groups are exemplified, for example, by vinyl, allyl, and but-3-en-1-yl. Included within this term are the cis and trans isomers or mixtures of these isomers.

“Substituted alkenyl” refers to alkenyl groups having from 1 to 3 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclyl, substituted heterocyclyl, heterocyclyloxy, substituted heterocyclyloxy, heterocyclylthio, substituted heterocyclylthio, nitro, SO₃H, sulfonyl, sulfonyloxy, thioacyl, thiol, alkylthio, and substituted alkylthio, wherein said substituents are as defined herein and with the proviso that any hydroxy substitution is not attached to a vinyl (unsaturated) carbon atom. In some embodiments, the alkenyl has 1 to 2 of the aforementioned groups.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

“Substituted alkvnyl” refers to alkynyl groups having from 1 to 3 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclyl, substituted heterocyclyl, heterocyclyloxy, substituted heterocyclyloxy, heterocyclylthio, substituted heterocyclylthio, nitro, SO₃H, sulfonyl, sulfonyloxy, thioacyl, thiol, alkylthio, and substituted alkylthio, wherein said substituents are as defined herein and with the proviso that any hydroxy or thiol substitution is not attached to a triply-bonded carbon atom. In some embodiments, the alkynyl has 1 to 2 of the aforementioned groups.

“Alkynyloxy” refers to the group —O-alkynyl, wherein alkynyl is as defined herein. Alkynyloxy includes, by way of example, ethynyloxy, propynyloxy, and the like.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclyl, and —C(O)O-substituted heterocyclyl, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“(Carboxyl ester)amino” refers to the groups —NR—C(O)O-alkyl, —NR—C(O)O-substituted alkyl, —NR—C(O)O-alkenyl, —NR—C(O)O-substituted alkenyl, —NR—C(O)O-alkynyl, —NR—C(O)O-substituted alkynyl, —NR—C(O)O-aryl, —NR—C(O)O-substituted aryl, —NR—C(O)O-cycloalkyl, —NR—C(O)O-substituted cycloalkyl, —NR—C(O)O-cycloalkenyl, —NR—C(O)O-substituted cycloalkenyl, —NR—C(O)O-heteroaryl, —NR—C(O)O-substituted heteroaryl, —NR—C(O)O-heterocyclyl, and —NR—C(O)O-substituted heterocyclyl, wherein R is alkyl or hydrogen and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“(Carboxyl ester)oxy” or “carbonate” refers to the groups—O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclyl, and —O—C(O)O-substituted heterocyclyl, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like.

“Cycloalkenyl” refers to non-aromatic cyclic alkyl groups of from 3 to 10 carbon atoms having single or multiple rings and having at least one double bond and preferably from 1 to 2 double bonds.

“Cycloalkynyl” refers to non-aromatic cycloalkyl groups of from 5 to 10 carbon atoms having single or multiple rings and having at least one triple bond.

“Cycloalkylene” refers to divalent cycloalkyl groups, wherein cycloalkyl is as defined herein.

“Substituted cycloalkylene” refers to cycloalkylene group having from 1 to 3 hydrogens replaced with substituents selected from the group consisting of alkyl, substituted alkyl, alkoxy, substituted alkoxy, acyl, acylamino, acyloxy, amino, substituted amino, aminocarbonyl, aryl, substituted aryl, aryloxy, substituted aryloxy, cyano, halogen, hydroxyl, nitro, carboxyl, carboxyl ester, cycloalkyl, substituted cycloalkyl, heteroaryl, substituted heteroaryl, heterocyclyl, substituted heterocyclyl, and oxo wherein said substituents are as defined herein. In some embodiments, the alkylene has 1 to 2 of the aforementioned groups. It is to be noted that when the cycloalkylene is substituted by an oxo group, 2 hydrogens attached to the same carbon of the cycloalkylene group are replaced by “═O”.

“Substituted cycloalkyl,” “substituted cycloalkenyl,” and “substituted cycloalkynyl” refer to a cycloalkyl, cycloalkenyl, or cycloalkynyl group having from 1 to 5 substituents selected from the group consisting of oxo, thioxo, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclyl, substituted heterocyclyl, heterocyclyloxy, substituted heterocyclyloxy, heterocyclylthio, substituted heterocyclylthio, nitro, SO₃H, sulfonyl, sulfonyloxy, thioacyl, thiol, alkylthio, and substituted alkylthio, wherein said substituents are as defined herein, provides that any hydroxy or thiol substitution is not attached to an unsaturated carbon atom. In some embodiments, the cycloalkyl or cycloalkenyl has 1 to 3 of the aforementioned groups. In some embodiments, the cycloalkyl group may have multiple condensed rings (e.g. tetrahydronaphthyl or tetrahydroanthacenyl), provided that the point of attachment is through an atom of the nonaromatic ring.

“Cycloalkoxy” refers to —O-cycloalkyl.

“Substituted cycloalkoxy” refers to —O-(substituted cycloalkyl).

“Cycloalkylthio” refers to —S-cycloalkyl. In other embodiments, sulfur may be oxidized to —S(O)— or —SO₂— moieties. The sulfoxide may exist as one or more stereoisomers.

“Substituted cycloalkylthio” refers to —S-(substituted cycloalkyl). In other embodiments, sulfur may be oxidized to —S(O)—, or —SO₂— moieties. The sulfoxide may exist as one or more stereoisomers.

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl. In other embodiments, sulfur may be oxidized to sulfinyl or sulfonyl moieties. The sulfoxide may exist as one or more stereoisomers.

“Substituted cycloalkenylthio” refers to —S-(substituted cycloalkenyl). In other embodiments, sulfur may be oxidized to —S(O)— or —SO₂— moieties. The sulfoxide may exist as one or more stereoisomers.

“Guanidino” refers to the group —NHC(═NH)NH₂.

“Substituted quanidino” refers to the group —NR³³C(═NR³³)N(R³³)₂, wherein each R³³ independently is selected from the group consisting of hydrogen, alkyl, substituted alkyl, aryl, substituted aryl, heteroaryl, substituted heteroaryl, heterocyclyl, and substituted heterocyclyl; two R groups attached to a common guanidino nitrogen atom are optionally joined together with the nitrogen bound thereto to form a heterocyclic or substituted heterocyclic group, provided that at least one R is not hydrogen; and said substituents are as defined herein. Two R³³ groups on distinct nitrogens are optionally joined together with the nitrogens bound thereto and the intervening carbon of the guanidine group to form a cyclic guanidine.

“Halo” or “halogen” refers to fluoro, chloro, bromo, and iodo and is preferably fluoro or chloro.

“Hydroxy” or “hydroxyl” refers to the group —OH.

“Heteroarvl” refers to an aromatic group of from 1 to 10 carbon atoms and 1 to 4 heteroatoms selected from the group consisting of oxygen, nitrogen, and sulfur within the ring. Such heteroaryl groups can have a single ring (e.g., pyridinyl or furyl) or multiple condensed rings (e.g., indolizinyl or benzothienyl), wherein the condensed rings may or may not be aromatic and/or contain a heteroatom, provided that the point of attachment is through an atom of the aromatic heteroaryl group. In one embodiment, the nitrogen and/or sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5 substituents selected from the group consisting of the same group of substituents defined for substituted aryl. In some embodiments, the heteroaryl has 1 to 3 of the aforementioned groups. In other embodiments, the heteroaryl has 1 to 2 of the aforementioned groups.

“Heteroarvloxy” refers to —O-heteroaryl.

“Substituted heteroaryloxy” refers to the group —O-(substituted heteroaryl).

“Heteroarvlthio” refers to the group —S-heteroaryl. In other embodiments, sulfur may be oxidized to —S(O)— or —SO₂— moieties. The sulfoxide may exist as one or more stereoisomers.

“Substituted heteroarylthio” refers to the group —S-(substituted heteroaryl). In other embodiments, sulfur may be oxidized to —S(O)— or —SO₂— moieties. The sulfoxide may exist as one or more stereoisomers.

“Heterocycle,” “heterocyclic,” “heterocycloalkyl” and “heterocyclyl” refer to a saturated or unsaturated group having a single ring or multiple condensed rings, including fused bridged and spiro ring systems, and having from 3 to 15 ring atoms, including 1 to 4 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In one embodiment, the nitrogen and/or sulfur atom(s) of the heterocyclic group are optionally oxidized to provide for the N-oxide, —S(O)—, or —SO₂— moieties.

“Substituted heterocyclic,” “substituted heterocycloalkyl,” and “substituted heterocyclyl” refer to heterocyclic groups that are substituted with from 1 to 5 of the same substituents as defined for substituted cycloalkyl. In some embodiments, the heterocyclyl has 1 to 3 of the aforementioned groups.

“Heterocyclyloxy” refers to the group —O-heterocyclyl.

“Substituted heterocyclyloxy” refers to the group —O-(substituted heterocyclyl).

“Heterocyclylthio” refers to the group —S-heterocyclyl. In other embodiments, sulfur may be oxidized to —S(O)— or —SO₂— moieties. The sulfoxide may exist as one or more stereoisomers.

“Substituted heterocvclylthio” refers to the group —S-(substituted heterocyclyl). In other embodiments, sulfur may be oxidized to —S(O)— or —SO₂— moieties. The sulfoxide may exist as one or more stereoisomers.

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

“Nitro” refers to the group —NO₂.

“Nitroso” refers to the group —NO.

“Oxo” refers to the atom (═O).

“Sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloal kenyl, —SO₂-substituted cycloalkenyl, —SO₂-aryl, —SO₂-substituted aryl, —SO₂-heteroaryl, —SO₂-substituted heteroaryl, —SO₂-heterocyclyl, and —SO₂-substituted heterocyclyl, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein. Sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclyl, and —OSO₂-substituted heterocyclyl, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclyl-C(S)—, and substituted heterocyclyl-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Thiol” refers to the group —SH.

“Thioxo” refers to the atom (═S).

“Alkylthio” refers to the group —S-alkyl, wherein alkyl is as defined herein. In other embodiments, sulfur may be oxidized to —S(O)—. The sulfoxide may exist as one or more stereoisomers.

“Substituted alkylthio” refers to the group —S-(substituted alkyl), wherein substituted alkyl is as defined herein. In other embodiments, sulfur may be oxidized to —S(O)—. The sulfoxide may exist as one or more stereoisomers.

“Phosphate” refers to a moiety selected from the group consisting of —OP(O)(OH)₂ (monophosphate or phospho), —OP(O)(OH)OP(O)(OH)₂ (diphosphate or diphospho) and —OP(O)(OH)OP(O)(OH)OP(O)(OH)₂ (triphosphate or triphospho) and salts thereof, including partial salts thereof.

“Phosphate ester” refers to a mono-, di- and tri-phosphate group, wherein one or more of the hydroxyl groups are replaced by an alkoxy group.

“Phosphonate” refers to a moiety selected from the group consisting of —OP(O)(R⁶)(OH) and —OP(O)(R⁶)(OR⁶) and salts thereof, including partial salts thereof, wherein each R⁶ independently of the others, is selected from hydrogen, alkyl, substituted alkyl, carboxylic acid, and carboxyl ester.

“Phosphorodiamidate” refers to

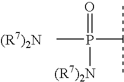

where each R⁷ may be the same or different and each is hydrogen, alkyl, substituted alkyl, cycloalkyl, or substituted cycloalkyl.

“Phosphoramidate monoester” refers to

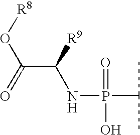

where R⁸ is hydrogen or alkyl and R⁹ is selected from the group consisting of hydrogen, alkyl, substituted alkyl, cycloalkyl, and substituted cycloalkyl.

“Phosphoramidate diester” refers to

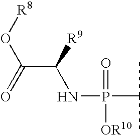

where R⁸ is hydrogen or alkyl, R¹⁰ is aryl, substituted aryl, alkyl, or substituted alkyl, and R⁹ is hydrogen, alkyl, substituted alkyl, cycloalkyl, or substituted cycloalkyl;

“Cyclic phosphoramidate” refers to

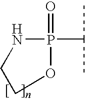

where n is 1 to 3.

“Cyclic phosphorodiamidate” refers to

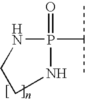

where n is 1 to 3.

“Phosphonamidate” refers to

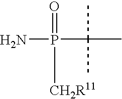

where R¹¹ is hydrogen, alkyl, substituted alkyl, cycloalkyl, or substituted cycloalkyl.

“Stereoisomer” and “stereoisomers” refer to compounds that have same atomic connectivity but different atomic arrangement in space. Stereoisomers include cis-trans isomers, E and Z isomers, enantiomers, and diastereomers.

“Syk Kinase” refers to the well-known 72 kDa non-receptor (cytoplasmic) spleen protein tyrosine kinase expressed in B-cells and other hematopoetic cells. Syk kinase includes two consensus Src-homology 2 (SH2) domains in tandem that bind to phosphorylated immunoreceptor tyrosine-based activation motifs (“ITAMs”), a “linker” domain and a catalytic domain (for a review of the structure and function of Syk kinase see Sada et al., 2001, J. Biochem. (Tokyo) 130:177-186); see also Turner et al., 2000, Immunology Today 21:148-154). Syk kinase has been extensively studied as an effector of B-cell receptor (BCR) signaling (Turner et al., 2000, supra). Syk kinase is also critical for tyrosine phosphorylation of multiple proteins that regulate important pathways leading from immunoreceptors, such as Ca2+ mobilization and mitogen-activated protein kinase (MAPK) cascades and degranulation. Syk kinase also plays a critical role in integrin signaling in neutrophils (see, e.g., Mocsai et al. 2002, Immunity 16:547-558).

As used herein, Syk kinase includes kinases from any species of animal, including but not limited to, Homo sapiens, simian, bovine, porcine, rodent, etc., recognized as belonging to the Syk family. Specifically included are isoforms, splice variants, allelic variants, and mutants, both naturally occurring and man-made. The amino acid sequences of such Syk kinases are well known and available from GENBANK. Specific examples of mRNAs encoding different isoforms of human Syk kinase can be found at GENBANK accession no. gi|34147655|ref|NM_{—}003177.3|[34147655], gi|496899|emb|Z29630.1|HSSYKPTK[496899] and gil|5030258|gb|BC011399.1|BC011399[15030258], which are incorporated herein by reference.

“Co-administration” can be in the form of a single formulation (combining, for example, a Syk kinase inhibitor and a thrombopoietin receptor agonist with pharmaceutically acceptable excipients, optionally segregating the two active ingredients in different excipient mixtures designed to independently control their respective release rates and durations) or by independent administration of separate formulations containing the active agents. “Co-administration” further includes concurrent administration (administration of a Syk kinase inhibitor and a thrombopoietin receptor agonist at the same time) and time varied administration (administration of the Syk kinase inhibitor at a time different from that of the thrombopoietin receptor agonist), as long as both the Syk kinase inhibitor and thrombopoietin receptor agonist are present in the body in therapeutically effective concentrations during at least partially overlapping times. When administered by independent administration of separate formulations containing the active agents, the formulations may be in the same or different formulations. For example, one of them may be administered in an oral formulation, the other may be administered intravenously. Other formulations that may be suitable are described herein.

The term “concurrent administration” means administering the agents substantially concurrently and encompasses administering the two agents in a single pharmaceutical dosage form and the administration of each active agent in its own separate pharmaceutical dosage formulation. Where separate dosage formulations are used, the agents can be administered at essentially the same time, i.e., concurrently.

The term “time varied administration” or “sequential administration” means administering the agents at separately staggered times. Thus, agents can be sequentially administered such that the beneficial pharmaceutical effects of the active agents are realized by the patient at substantially the same time. For example, the interval between administering a formulation of one of the active ingredients and a formulation of the other active ingredient can be about 30 minutes, about 1 hour, about 2 hours, about 3 hours, about 4 hours, etc., or a suitable interval in between.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

“Patient” refers to human and non-human animals, especially mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like.

“Prodrug” refers to a derivative of an active 4-pyrimidineamine compound (drug) that may require a transformation under the conditions of use, such as within the body, to release the active 2,4-pyrimidinediamine drug. Prodrugs are frequently, but not necessarily, pharmacologically inactive until converted into the active drug. Prodrugs are typically obtained by masking one or more functional groups in an active 2,4-pyrimidinediamine drug believed to be in part required for activity with a progroup (defined below) to form a promoiety which undergoes a transformation, such as cleavage, under the specified conditions of use to release the functional group, and hence the active 2,4-pyrimidinediamine drug. The cleavage of the promoiety may proceed spontaneously, such as by way of a hydrolysis reaction, or it can be catalyzed or induced by another agent, such as an enzyme, light, an acid or base, or a change of or exposure to a physical or environmental parameter, such as temperature. The agent can be endogenous to the conditions of use, such as an enzyme present in the cells to which the prodrug is administered or the acidic conditions of the stomach, or it can be supplied exogenously.

“Progroup” refers to a type of protecting group that, when used to mask a functional group within an active 2,4-pyrimidinediamine drug to form a promoiety, converts the drug into a prodrug. Progroups are typically attached to the functional group of the drug via bonds that are cleavable under specified conditions of use. Thus, a progroup is that portion of a promoiety that cleaves to release the functional group under the specified conditions of use. As a specific example, an amide promoiety of the formula —NH—C(O)CH₃ comprises the progroup —C(O)CH₃.

“Pharmaceutically effective amount” and “therapeutically effective amount” refer to an amount of a compound sufficient to treat a specified disorder or disease or one or more of its symptoms and/or to prevent the occurrence of the disease or disorder. In reference to tumorigenic proliferative disorders, a pharmaceutically or therapeutically effective amount comprises an amount sufficient to, among other things, cause the tumor to shrink or decrease the growth rate of the tumor.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate is referred to as a “hydrate.”

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

Further, as used herein, the term “optionally substituted alkyl,” for example, is used interchangeably with “alkyl” or “substituted alkyl.” Similarly, “optionally substituted aryl” is used interchangeably with “aryl” or “substituted aryl”; “optionally substituted heteroaryl” is used interchangeably with “heteroaryl” or “substituted heteroaryl,” etc.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups are limited to-substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are easily recognized by a person having ordinary skill in the art.

### B. Methods of the Invention

In one aspect, the invention provides methods of treating thrombocytopenia comprising co-administering to a patient in need thereof a Syk kinase inhibitor and a thrombopoietin receptor agonist. Another aspect of the invention provides methods of increasing platelet levels in a patient having or at risk for immune thrombocytopenia comprising co-administering a Syk kinase inhibitor and a thrombopoietin receptor agonist.

Thrombocytopenia (platelet deficiencies) may be present for various reasons, including chemotherapy and other therapy with a variety of drugs, radiation therapy, surgery, accidental blood loss, and other specific disease conditions. Exemplary specific disease conditions that involve thrombocytopenia and may be treated in accordance with this invention are: aplastic anemia; idiopathic or immune thrombocytopenia (ITP), including idiopathic thrombocytopenic purpura; HIV associated ITP and HIV-related thrombotic thrombocytopenic purpura; metastatic tumors which result in thrombocytopenia; systemic lupus erythematosus; including neonatal lupus syndrome splenomegaly; Fanconi's syndrome; vitamin B12 deficiency; folic acid deficiency; May-Hegglin anomaly; Wiskott-Aldrich syndrome; chronic liver disease; myelodysplastic syndrome associated with thrombocytopenia; paroxysmal nocturnal hemoglobinuria; acute profound thrombocytopenia following C7E3 Fab (Abciximab) therapy; alloimmune thrombocytopenia, including maternal alloimmune thrombocytopenia; thrombocytopenia associated with antiphospholipid antibodies and thrombosis; autoimmune thrombocytopenia; drug-induced immune thrombocytopenia, including carboplatin-induced thrombocytopenia, heparin-induced thrombocytopenia; fetal thrombocytopenia; gestational thrombocytopenia; Hughes' syndrome; lupoid thrombocytopenia; accidental and/or massive blood loss; myeloproliferative disorders; thrombocytopenia in patients with malignancies; thrombotic thrombocytopenia purpura, including thrombotic microangiopathy manifesting as thrombotic thrombocytopenic purpura/hemolytic uremic syndrome in cancer patients; autoimmune hemolytic anemia; occult jejunal diverticulum perforation; pure red cell aplasia; autoimmune thrombocytopenia; nephropathia epidemica; rifampicin-associated acute renal failure; Paris-Trousseau thrombocytopenia; neonatal alloimmune thrombocytopenia; paroxysmal nocturnal hemoglobinuria; hematologic changes in stomach cancer; hemolytic uremic syndromes in childhood; hematologic manifestations related to viral infection including hepatitis A virus and CMV-associated thrombocytopenia. Also, certain treatments for AIDS result in thrombocytopenia (e.g., AZT). Certain wound healing disorders might also benefit from an increase in platelet numbers.

As used herein, the term “thrombopoietin receptor agonist” refers to a compound capable of binding to and activating the thrombopoietin receptor (also known as the c-MP1 receptor), thus stimulating production of platelets and their precursor cells, such as megakaryocytes. Thus, the thrombopoietin receptor agonists of use in the present invention have thrombopoietic activity, i.e., the ability to stimulate, in vivo and in vitro, the production of platelets, and/or megakaryocytopoietic activity, i.e., the ability to stimulate, in vivo and in vitro, the production of platelet precursors.

Various thrombopoietin receptor agonists are known in the art. Thrombopoietin receptor agonists can take the form of peptide mimetics of thrombopoietin, as described in international patent publications, WO 96/4018, WO 96/40750, and WO 98/25965, incorporated herein by reference. In one implementation of the invention, the thrombopoietin receptor agonist is a polypeptide, preferably thrombopoietin itself or a peptide mimetic of thrombopoietin. In certain instances, the peptide mimetic of thrombopoietin will not share any amino acid sequence homology or identity with thrombopoietin. In a preferred implementation, the thrombopoietin receptor agonist is a polypeptide comprising the sequence Ile-Glu-Gly-Pro-Thr-Leu-Arg-Gln-Trp-Leu-Ala-Ala-Arg-Ala (SEQ ID NO: 1). In another preferred implementation, the thrombopoietin receptor agonist is the polypeptide, AMG531 (Amgen).

A variety of small molecule thrombopoietin receptor agonists also have been described. These include 1,4-benzodiazepin-2-ones (JP11001477), metal complexes derived from Schiff base ligands (WO 99/11262), cyclic polyamine derivatives (WO 00/28987), thiazol-2-yl-benzamides (WO 01/07423, WO 01/53267), azo-aryl derivatives (WO 00/35446, WO 1/17349), 2-aryl-naphthimidazoles (WO 01/39773, WO 01/53267), semicarbazone derivatives (WO 01/34585), and substituted thiosemicarbazone derivatives (U.S. Pat. No. 7,241,783). Each of the foregoing patent publications is incorporated herein by reference. In a preferred implementation of the invention, the thrombopoietin receptor agonist is a small molecule, such as eltrombopag (GlaxoSmithKline) or 3′-{N′-[1-(3,4-dimethylphenyl)-3-methyl-5-oxo-1,5-di-hydropyrazol-4-ylidene]hydrazine}-2′-hydroxybiphenyl-3-carboxylic acid, or a pharmaceutically acceptable salt or ester thereof.

Inhibition of Syk kinase can be determined by an assay, such as the in vitro CHMC or BMMC and other in vitro assays described in U.S. application Ser. No. 10/355,543 filed Jan. 31, 2003 (US2004/0029902A1), international application Serial No. PCT/US03/03022 filed Jan. 31, 2003 (WO 03/063794), U.S. application Ser. No. 10/631,029 filed Jul. 29, 2003, international application Serial No. PCT/US03/24087 (WO2004/014382), U.S. application Ser. No. 10/903,263 (US2005/0234049A1) filed Jul. 30, 2004, and international application Serial No. PCT/US2004/24716 (WO005/016893).

Immune Thrombocytopenic Purpura (ITP) is an autoimmune disease characterized by the destruction of platelets by pathogenic autoantibodies. Clearance of platelets by phagocytic cells such as macrophages occurs through activating receptors for the Fc portion of IgG (FcγR). Aggregation of the Fc receptors, induced by antibody-antigen complexes, can induce a multitude of cellular functions including degranulation, arachidonic acid metabolism, antibody dependent cellular cytotoxicity (ADCC), phagocytosis, and cytokine secretion, depending on the cell type. These products lead to tissue damage and propagation of inflammatory responses, and thus inhibition of Fc-mediated signaling may diminish the ability of neutrophils and macrophages to affect end-organ damage in diseases involving immune complex activation. The intracellular protein tyrosine kinase Syk is a key mediator of FcR signalling, and it has been demonstrated that macrophages deficient in Syk are defective in phagocytosis of particles bound by FcR. Furthermore, FcRγ- or FcγRIII-deficient mice were protected from the pathogenic activity of antiplatelet antibodies (Clynes and Ravetch, 1995; Samuelsson et al., 2001).

Syk kinase inhibitors that are useful in the therapeutic combination of the invention preferably are 2,4 pyrimidinediamine compounds having the general formula I:

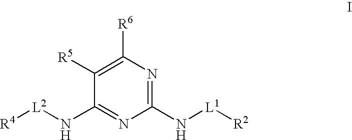

a salt, hydrate, solvate, N-oxide or prodrug thereof, wherein:
L¹ and L², independently of each other, are selected from the group consisting of a direct bond, a (C1-C3) alkylene optionally substituted with one or more of the same or different R⁹ groups, and 1-3 membered heteroalkyldiyl optionally substituted with one or more of the same or different R⁹ groups;
R² is selected from the group consisting of (C1-C6) alkyl optionally substituted with one or more of the same or different R⁸ groups, (C2-C6) alkenyl optionally substituted with one or more of the same or different R⁸ groups, (C2-C6) alkynyl optionally substituted with one or more of the same or different R⁸ groups, (C3-C8) cycloalkyl optionally substituted with one or more of the same or different R⁸ groups, 3-8 membered heterocyclyl optionally substituted with one or more of the same or different R⁸ groups, (C5-C15) aryl optionally substituted with one or more of the same or different R⁸ groups, and 5-15 membered heteroaryl optionally substituted with one or more of the same or different R⁸ groups;
R⁴ is selected from the group consisting of hydrogen, (C1-C6) alkyl optionally substituted with one or more of the same or different R⁸ groups, (C2-C6) alkenyl optionally substituted with one or more of the same or different R⁸ groups, (C2-C6) alkynyl optionally substituted with one or more of the same or different R⁸ groups, (C3-C8) cycloalkyl optionally substituted with one or more of the same or different R⁸ groups, 3-8 membered heterocyclyl optionally substituted with one or more of the same or different R⁸ groups, (C5-C15) aryl optionally substituted with one or more of the same or different R⁸ groups, and 5-15 membered heteroaryl optionally substituted with one or more of the same or different R⁸ groups;
R⁵ is selected from the group consisting of R⁶, (C2-C6) alkenyl optionally substituted with one or more of the same or different R⁸ groups, (C2-C6) alkynyl optionally substituted with one or more of the same or different R⁸ groups, and (C3-C8) cycloalkyl optionally substituted with one or more of the same or different R⁸ groups;
each R⁶, independently of the others, is selected from the group consisting of hydrogen, —OR^{d}, —SR^{d}, (C1-C3) haloalkyloxy, (C1-C3) perhaloalkyloxy, —NR^{c}R^{c}, halogen, (C1-C3) haloalkyl, (C1-C3) perhaloalkyl, —CF₃, —CH₂CF₃, —CF₂CF₃, —CN, —NC, —OCN, —SCN, —NO, —NO₂, —N₃, —S(O)R^{d}, —S(O)₂R^{d}, —S(O)NR^{c}R^{c}, —S(O)₂NR^{c}R^{c}, —OS(O)R^{d}, —OS(O)₂R^{d}, —OS(O)₂OR^{d}, —OS(O)NR^{c}R^{c}, —OS(O)₂NR^{c}R^{c}, —C(O)R^{d}, —C(O)OR^{d}, —C(O)NR^{c}R^{c}, —C(NH)NR^{c}R^{c}, —OC(O)R^{d}, —SC(O)R^{d}, —OC(O)OR^{d}, —SC(O)OR^{d}, —OC(O)NR^{c}R^{c}, —SC(O)NR^{c}R^{c}, —OC(NH)NR^{c}R^{c}, —SC(NH)NR^{c}R^{c}, —[NHC(O)]ₙR^{d}, —[NHC(O)]ₙOR^{d}, —[NHC(O)]ₙR^{c}R^{c}, —[NHC(NH)]ₙR^{c}R^{c}, (C5-C10) aryl optionally substituted with one or more of the same or different R⁸ groups, (C6-C16) arylalkyl optionally substituted with one or more of the same or different R⁸ groups, 5-10 membered heteroaryl optionally substituted with one or more of the same or different R⁸ groups, and 6-16 membered heteroarylalkyl optionally substituted with one or more of the same or different R⁸ groups;
R⁸ is selected from the group consisting of R^{e}, R^{b}, R^{e} substituted with one or more of the same or different R^{a} or R^{b}, —OR^{a} substituted with one or more of the same or different R^{a} or R^{b}, —B(OR^{a})₂, —B(NR^{c}R^{c})₂, —(CH₂)ₘR^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂, —O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘR^{b}, —C(O)NH—(CHR^{a})ₘR^{b}, —O(CH₂)ₘC(O)NH—(CH₂)ₘR^{b}, —S—(CH₂)ₘ—C(O)NH—(CH₂)ₘ—R^{b}, —O—(CHR^{a})ₘ—C(O)NH—(CHR^{a})—R^{b}, —S—(CHR^{a})ₙC(O)NH—(CH₂)ₘ—R^{b}, —NH—(CHR^{a})ₘ—R^{b}, —N[(CH₂)ₘR^{b}]₂, —NH—C(O)—NH—(CH₂)ₘ—R^{b}, —NH—C(O)—(CH₂)ₘ—CHR^{b}R^{b} and —NH—(CH₂)ₘ—C(O)—NH—(CH₂)ₘ—R^{b};
each R⁹, independently of the others, is selected from the group consisting of (C1-C6) alkyl, —OR^{a}, —C(O)OR^{a}, (C5-C10) aryl optionally substituted with one or more of the same or different halogens, phenyl optionally substituted with one or more of the same or different halogens, and 5-10 membered heteroaryl optionally substituted with one or more of the same or different halogens;
each R^{a}, independently of the others, is selected from the group consisting of hydrogen, (C1-C6) alkyl, (C3-C8) cycloalkyl, (C4-C11) cycloalkylalkyl, (C5-C10) aryl, (C6-C16) arylalkyl, 2-6 membered heteroalkyl, 3-8 membered heterocyclyl, 4-11 membered heterocyclylalkyl, 5-10 membered heteroaryl and 6-16 membered heteroarylalkyl;
each R^{b}, independently of the others, is a suitable group selected from the group consisting of ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂R^{c}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}—C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c}, independently of the others, is a progroup or R^{a}, or, alternatively, two R^{c} are taken together with the nitrogen atom to which they are bonded to form a 5 to 8-membered heterocyclyl or heteroaryl which may optionally include one or more of the same or different additional heteroatoms and which may optionally be substituted with one or more of the same or different R^{a} or suitable R^{b} groups;
each R^{d}, independently of the others, is a progroup or R^{a};
each R^{e}, independently of the others, is selected from the group consisting of (C1-C6) alkyl, (C3-C8) cycloalkyl, (C4-C11) cycloalkylalkyl, (C5-C10) aryl, (C6-C16) arylalkyl, 2-6 membered heteroalkyl, 3-8 membered heterocyclyl, 4-11 membered cycloheteroalkylalkyl, 5-10 membered heteroaryl and 6-16 membered heteroarylalkyl;
each m, independently of the others, is an integer from 1 to 3; and
each n, independently of the others, is an integer from 0 to 3.

In a particularly preferred embodiment, the Syk kinase inhibitor is a compound of formula II:

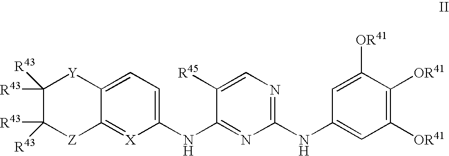

or a pharmaceutically acceptable salt or N-oxide thereof, wherein
X is selected from the group consisting of N and CH;
Y is selected from the group consisting of O, S, SO, SO₂, SONR⁴¹, NH, and NR⁴²;
Z is selected from the group consisting of O, S, SO, SO₂, SONR⁴¹, NH, and NR⁴²;
each R⁴¹, independently of the others, is hydrogen or lower alkyl;
each R⁴², independently of the others, is selected from the group consisting of alkyl, substituted alkyl, alkenyl, substituted alkenyl, alkynyl, substituted alkynyl, cycloalkynyl, substituted cycloalkynyl, aryl, substituted aryl, cycloalkyl, substituted cycloalkyl, heteroaryl, substituted heteroaryl, heterocyclyl, substituted heterocyclyl, hydroxyl, alkoxy, substituted alkoxy, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxyl ester, (carboxyl ester)oxy, and —(CRR)ₙ—R^{p}, wherein
   each R, independently of the others, is selected from the group consisting of hydrogen, lower alkyl and halo;
   n is an integer from 0 to 4; and
   R^{p} is selected from the group consisting of phosphate, phosphate ester, phosphonate, phosphorodiamidate, phosphoramidate monoester, phosphoramidate diester, cyclic phosphoramidate, cyclic phosphorodiamidate, phosphonamidate, and cyclic phosphonamidate;
each R⁴³, independently of the others, is selected from the group consisting of hydrogen, alkyl, substituted alkyl, alkoxy, substituted alkoxy, alkenyl, substituted alkenyl, alkynyl, substituted alkynyl, cycloalkynyl, substituted cycloalkynyl, alkynyloxy, amino, substituted amino, aryl, substituted aryl, aryloxy, substituted aryloxy, cyano, cycloalkyl, substituted cycloalkyl, cycloalkoxy, substituted cycloalkoxy, heteroaryl, substituted heteroaryl, heteroaryloxy, substituted heteroaryloxy, heterocyclyl, substituted heterocyclyl, heterocyclyloxy, substituted heterocyclyloxy, aminocarbonyl, aminocarbonyloxy, carboxyl, carboxyl ester, (carboxyl ester)oxy, nitro, and halo, or, alternatively, two R⁴³ bonded to the same carbon atom are taken together to form an oxo (═O), ═NH or ═NR⁴⁴ group and the other two R⁴³ are as defined above;
each R⁴⁴, independently of the others, is selected from the group consisting of (C1-C6) alkyl and (C5-C14) aryl; and
R⁴⁵ is selected from the group consisting of alkyl, substituted alkyl, alkoxy, substituted alkoxy, amino, substituted amino, carboxyl, carboxyl ester, cyano, halo, nitro, alkenyl, substituted alkenyl, alkynyl and substituted alkynyl.

More preferably, the Syk kinase inhibitor is a compound of formula II, wherein two R⁴³ bonded to the same carbon atom are taken together to form an oxo (═O) group and the other two R⁴³ independently of one another are hydrogen or alkyl. Also preferred, the Syk kinase inhibitor is a compound of formula II, wherein X is N. In another preferred implementation of formula II, each R⁴¹ is methyl. In a particularly preferred implementation, the Syk kinase inhibitor is a compound of formula II, wherein Y is O and Z is HN or NR⁴², preferably wherein R⁴² is —(CRR)ₙ—R^{p}, more preferably where n is one, each R is hydrogen, and R^{p} is phosphate or phosphate ester.

While not being bound by a theory of the invention, Syk kinase inhibitors are believed to elicit their pro-thrombotic effects by inhibiting immune-targeted destruction of platelets.

An important tyrosine kinase involved in the signal transduction pathways associated with crosslinking the FcεRI and/or FcγRI, as well as other signal transduction cascades, is Syk kinase (see Valent et al., 2002, Intl. J. Hematol. 75(4):257-362 for review). Crosslinking of Fc receptors, such as the high affinity receptor for IgE (FcεRI) and/or the high affinity receptor for IgG (FcγRI) activates a signaling cascade in mast, basophil and other immune cells that results in the release of chemical mediators responsible for numerous adverse events. For example, such crosslinking leads to the release of preformed mediators of Type I (immediate) anaphylactic hypersensitivity reactions, such as histamine, from storage sites in granules via degranulation. It also leads to the synthesis and release of other mediators, including leukotrienes, prostaglandins and platelet-activating factors (PAFs) that play important roles in inflammatory reactions. Additional mediators that are synthesized and released upon crosslinking Fc receptors include cytokines and nitric oxide. Biochemical data confirm that Syk kinase inhibitors, such as the preferred 2,4-pyrimidinediamine compounds, exert their degranulation inhibitory effect, at least in part, by blocking or inhibiting the signal transduction cascade(s) initiated by crosslinking of the high affinity Fc receptors for IgE (FcεRI) and/or IgG (FcγRI). Indeed, the 2,4-pyrimidinediamine compounds are potent inhibitors of both FcεRI—-mediated and FcεRI-mediated degranulation. The methods also permit the regulation of, and in particular the inhibition of, downstream processes that result as a consequence of activating such Fc receptor signaling cascade(s). Such downstream processes include, but are not limited to, FcεRI-mediated and/or FcγRI-mediated degranulation, cytokine production and/or the production and/or release of lipid mediators such as leukotrienes and prostaglandins.

The preferred 2,4-pyrimidinediamine compounds and prodrugs of the invention can be synthesized via a variety of different synthetic routes using commercially available starting materials and/or starting materials prepared by conventional synthetic methods. Suitable exemplary methods that can be routinely adapted to synthesize the 2,4-pyrimidinediamine compounds and prodrugs of the invention are found in U.S. Pat. No. 5,958,935, the disclosure of which is incorporated herein by reference. Specific examples describing the synthesis of numerous 2,4-pyrimidinediamine compounds and prodrugs, as well as intermediates therefor, are described in U.S. Patent Application Publication US2004/0029902A1, the contents of which are incorporated herein by reference. Suitable exemplary methods that can be routinely used and/or adapted to synthesize active 2,4-pyrimidinediamine compounds can also be found in international patent applications WO 03/063794, WO2004/014382, and WO005/016893, U.S. Patent Application Publications US2007/0060603A1 and US2005/0234049A1, the disclosures of which are incorporated herein by reference. All of the compounds described herein (including prodrugs) can be prepared by routine adaptation of these methods.

Myriad references teaching methods useful for synthesizing pyrimidines generally, as well as starting materials, are known in the art. For specific guidance, the reader is referred to Brown, D. J., “The Pyrimidines”, in The Chemistry of Heterocyclic Compounds, Volume 16 (Weissberger, A., Ed.), 1962, Interscience Publishers, (A Division of John Wiley & Sons), New York (“Brown I”); Brown, D. J., “The Pyrimidines”, in The Chemistry of Heterocyclic Compounds, Volume 16, Supplement I (Weissberger, A. and Taylor, E. C., Ed.), 1970, Wiley-Interscience, (A Division of John Wiley & Sons), New York (Brown II”); Brown, D. J., “The Pyrimidines”, in The Chemistry of Heterocyclic Compounds, Volume 16, Supplement II (Weissberger, A. and Taylor, E. C., Ed.), 1985, An Interscience Publication (John Wiley & Sons), New York (“Brown III”); Brown, D. J., “The Pyrimidines” in The Chemistry of Heterocyclic Compounds, Volume 52 (Weissberger, A. and Taylor, E. C., Ed.), 1994, John Wiley & Sons, Inc., New York, pp. 1-1509 (Brown IV”); Kenner, G. W. and Todd, A., in Heterocyclic Compounds, Volume 6, (Elderfield, R. C., Ed.), 1957, John Wiley, New York, Chapter 7 (pyrimidines); Paquette, L. A., Principles of Modern Heterocyclic Chemistry, 1968, W. A. Benjamin, Inc., New York, pp. 1-401 (uracil synthesis pp. 313, 315; pyrimidinediamine synthesis pp. 313-316; amino pyrimidinediamine synthesis pp. 315); Joule, J. A., Mills, K. and Smith, G. F., Heterocyclic Chemistry, 3^{rd} Edition, 1995, Chapman and Hall, London, UK, pp. 1-516; Vorbrüggen, H. and Ruh-Pohlenz, C., Handbook of Nucleoside Synthesis, John Wiley & Sons, New York, 2001, pp. 1-631 (protection of pyrimidines by acylation pp. 90-91; silylation of pyrimidines pp. 91-93); Joule, J. A., Mills, K. and Smith, G. F., Heterocyclic Chemistry, 4^{th} Edition, 2000, Blackwell Science, Ltd, Oxford, UK, pp. 1-589; and Comprehensive Organic Synthesis, Volumes 1-9 (Trost, B. M. and Fleming, I., Ed.), 1991, Pergamon Press, Oxford, UK.

Those of skill in the art will appreciate that the 2,4-pyrimidinediamine compounds described herein may include functional groups that can be masked with progroups to create prodrugs. Exemplary prodrugs of 2,4-pyrimidinediamine compounds are described in U.S. Patent Application Publication US2006/0211657A1, which is incorporated herein by reference. Such prodrugs are usually, but need not be, pharmacologically inactive until converted into their active drug form. Indeed, many of the 2,4-pyrimidinediamine compounds described in this invention include promoieties that are hydrolyzable or otherwise cleavable under conditions of use. For example, ester groups commonly undergo acid-catalyzed hydrolysis to yield the parent carboxylic acid when exposed to the acidic conditions of the stomach, or base-catalyzed hydrolysis when exposed to the basic conditions of the intestine or blood. Thus, when administered to a subject orally, 2,4-pyrimidinediamine compounds that include ester moieties can be considered prodrugs of their corresponding carboxylic acid, regardless of whether the ester form is pharmacologically active.

The mechanism by which the progroup(s) metabolizes is not critical, and can be caused by, for example, hydrolysis under the acidic conditions of the stomach, as described above, and/or by enzymes present in the digestive tract and/or tissues or organs of the body. Indeed, the progroup(s) can be selected to metabolize at a particular site within the body. For example, many esters are cleaved under the acidic conditions found in the stomach. Prodrugs designed to cleave chemically in the stomach to the active 2,4-pyrimidinediamine can employ progroups including such esters. Alternatively, the progroups can be designed to metabolize in the presence of enzymes such as esterases, amidases, lipolases, phosphatases including ATPases and kinase etc. Progroups including linkages capable of metabolizing in vivo are well known, and include, by way of example and not limitation, ethers, thioethers, silylethers, silylthioethers, esters, thioesters, carbonates, thiocarbonates, carbamates, thiocarbamates, ureas, thioureas, carboxamides, etc. In some instances, a “precursor” group that is oxidized by oxidative enzymes such as, for example, cytochrome P450 of the liver, to a metabolizable group, can be selected.

In the prodrugs, any available functional moiety can be masked with a progroup to yield a prodrug. Functional groups within the 2,4-pyrimidinediamine compounds that can be masked with progroups for inclusion in a promoiety include, but are not limited to, amines (primary and secondary), hydroxyls, sulfanyls (thiols), carboxyls, etc. Myriad progroups suitable for masking such functional groups to yield promoieties that are cleavable under the desired conditions of use are known in the art. All of these progroups, alone or in combinations, can be included in the prodrugs.

In some embodiments of the 2,4-pyrimidinediamine compounds and methods of using the compounds, the progroup(s) can be attached to any available primary or secondary amine, including, for example, the N2 nitrogen atom of the 2,4-pyrimidinediamine moiety, the N4 nitrogen atom of the 2,4-pyrimidinediamine moiety, and/or a primary or secondary nitrogen atom included in a substituent on the 2,4-pyrimidinediamine compound.

In particular embodiments of the 2,4-pyrimidinediamine compounds and methods of using the compounds, the prodrugs described herein are 2,4-pyrimidinediamine compounds that are substituted at the N4 nitrogen of the 2,4-pyrimidinediamine moiety with a substituted or unsubstituted nitrogen-containing bicyclic ring that includes at least one progroup at one or more of: the nitrogen atom(s) of the bicyclic ring, the N2 nitrogen of the 2,4-pyrimidinediamine moiety, and/or the N4 nitrogen of the 2,4-pyrimidinediamine moiety.

As noted above, the identity of the progroup is not critical, provided that it can be metabolized under the desired conditions of use, for example under the acidic conditions found in the stomach and/or by enzymes found in vivo, to yield a the biologically active group, e.g., the 2,4-substituted pyrimidinediamines as described herein. Thus, skilled artisans will appreciate that the progroup can comprise virtually any known or later-discovered hydroxyl, amine or thiol protecting group. Non-limiting examples of suitable protecting groups can be found, for example, in Protective Groups in Organic Synthesis, Greene & Wuts, 2nd Ed., John Wiley & Sons, New York, 1991 (especially pages 10-142 (alcohols, 277-308 (thiols) and 309-405 (amines) the disclosure of which is incorporated herein by reference).

Additionally, the identity of the progroup(s) can also be selected so as to impart the prodrug with desirable characteristics. For example, lipophilic groups can be used to decrease water solubility and hydrophilic groups can be used to increase water solubility. In this way, prodrugs specifically tailored for selected modes of administration can be obtained. The progroup can also be designed to impart the prodrug with other properties, such as, for example, improved passive intestinal absorption, improved transport-mediated intestinal absorption, protection against fast metabolism (slow-release prodrugs), tissue-selective delivery, passive enrichment in target tissues, targeting-specific transporters, etc. Groups capable of imparting prodrugs with these characteristics are well-known, and are described, for example, in Ettmayer et al., 2004, J. Med. Chem. 47(10):2393-2404, the disclosure of which is incorporated by reference. All of the various groups described in these references can be utilized in the prodrugs described herein.

### C. Pharmaceutical Compositions of the Invention

The invention also provides pharmaceutical compositions comprising a Syk kinase inhibitor and a thrombopoietin receptor agonist. Pharmaceutical compositions comprising the Syk kinase inhibitors, preferably the 2,4-pyrimidinediamine compounds, and/or the thrombopoietin receptor agonists described herein can be manufactured by means of conventional mixing, dissolving, granulating, dragee-making levigating, emulsifying, encapsulating, entrapping or lyophilization processes. The compositions can be formulated in conventional manner using one or more physiologically acceptable carriers, diluents, excipients or auxiliaries which facilitate processing of the active compounds into preparations which can be used pharmaceutically.

The Syk kinase inhibitor and/or thrombopoietin receptor agonist can be formulated in the pharmaceutical compositions per se, or in the form of a hydrate, solvate, N-oxide or pharmaceutically acceptable salt, as described herein. Typically, such salts are more soluble in aqueous solutions than the corresponding free acids and bases, but salts having lower solubility than the corresponding free acids and bases may also be formed.

In one embodiment, this invention provides a pharmaceutical formulation comprising a compound selected from the compounds of the invention, as described herein, or a prodrug thereof, and at least one pharmaceutically acceptable excipient, diluent, preservative, or stabilizer, or mixtures thereof.

In another embodiment, the method can be practiced as a therapeutic approach towards the treatment of the conditions described herein. Thus, in a specific embodiment, the Syk kinase inhibitor and/or thrombopoietin receptor agonist (and the various forms described herein, including pharmaceutical formulations comprising the compounds (in the various forms)) can be used to treat thrombocytopenia in animal subjects, including humans. The methods generally comprise administering to the subject an amount of a Syk kinase inhibitor and thrombopoietin receptor agonist, or a salt, prodrug, hydrate or N-oxide thereof, effective to treat and/or prevent thrombocytopenia. In one embodiment, the subject is a mammal, including, but not limited to, bovine, horse, feline, canine, rodent, or primate. In another embodiment, the subject is a human.

The compounds can be provided in a variety of formulations and dosages. It is contemplated that a typical dosage of a Syk kinase inhibitor that is co-administered with a thrombopoietin receptor agonist will range from about 0.001 mg/kg to about 1000 mg/kg, about 0.01 mg/kg to about 100 mg/kg, or from about 0.1 mg/kg to about 10 mg/kg.

The compounds can be provided in a pharmaceutically acceptable form including, where the compound or prodrug can be formulated in the pharmaceutical compositions per se, or in the form of a hydrate, solvate, N-oxide or pharmaceutically acceptable salt, as described herein. Typically, such salts are more soluble in aqueous solutions than the corresponding free acids and bases, but salts having lower solubility than the corresponding free acids and bases may also be formed. It is to be understood that reference to 2,4-pyrimidinediamine compound in discussions of formulations is also intended to include, where appropriate as known to those of skill in the art, formulation of prodrugs of the 2,4-pyrimidinediamine compounds, disclosed herein. Preferably, the Syk kinase inhibitor will be provided as a sodium salt, potassium salt, calcium salt, magnesium salt, arginine salt or lysine salt. In each case, the pharmaceutically acceptable salt can comprise multiple counter ions, e.g. to form a di-sodium salt or di-potassium salt.

In one embodiment, the compounds are provided as non-toxic pharmaceutically acceptable salts, as noted previously. Suitable pharmaceutically acceptable salts of the compounds of this invention include acid addition salts such as those formed with hydrochloric acid, fumaric acid, p-toluenesulphonic acid, maleic acid, succinic acid, acetic acid, citric acid, tartaric acid, carbonic acid or phosphoric acid. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The pharmaceutically acceptable salts of the present invention can be formed by conventional means, such as by reacting the free base form of the product with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention includes within its scope solvates of the Syk kinase inhibitor and/or thrombopoietin receptor agonist compounds and salts thereof, for example, hydrates.

The Syk kinase inhibitors and thrombopoietin receptor agonists can be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients and vehicles appropriate for each route of administration. In addition to the treatment of warm-blooded animals such as mice, rats, horses, cattle, sheep, dogs, cats, monkeys, etc., the compounds of the invention can be effective in humans.

The pharmaceutical compositions for the administration of Syk kinase inhibitors and thrombopoietin receptor agonists may conveniently be presented in dosage unit form and can be prepared by any of the methods well known in the art of pharmacy. The pharmaceutical compositions can be, for example, prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired therapeutic effect. For example, pharmaceutical compositions of the invention may take a form suitable for virtually any mode of administration, including, for example, topical, ocular, oral, buccal, systemic, nasal, injection, transdermal, rectal, vaginal, etc., or a form suitable for administration by inhalation or insufflation.

For topical use, creams, ointments, jellies, gels, solutions or suspensions, etc., containing the Syk kinase inhibitor sand/or thrombopoietin receptor agonists can be employed. In certain embodiments, Syk kinase inhibitor and/or thrombopoietin receptor agonist can be formulated for topical administration with polyethylene glycol (PEG). These formulations may optionally comprise additional pharmaceutically acceptable ingredients such as diluents, stabilizers and/or adjuvants.

According to the invention, 2,4-pyrimidinediamine compounds can be used for manufacturing a composition or medicament, including medicaments suitable for topical administration. The invention also relates to methods for manufacturing compositions including 2,4-pyrimidinediamine compounds in a form that is suitable for topical administration.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, dextrose solution, etc., before use. To this end, the active compound(s) can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art with, for example, sugars, films or enteric coatings. Additionally, the pharmaceutical compositions containing the Syk kinase inhibitor and/or thrombopoietin receptor agonist as active ingredient or prodrug thereof in a form suitable for oral use, may also include, for example, troches, lozenges, aqueous or oily suspensions, dispersible powders or granules, emulsions, hard or soft capsules, or syrups or elixirs. Compositions intended for oral use can be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient (including prodrug) in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch, or alginic acid); binding agents (e.g. starch, gelatin or acacia); and lubricating agents (e.g. magnesium stearate, stearic acid or talc). The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques described in the U.S. Pat. Nos. 4,256,108; 4,166,452; and 4,265,874 to form osmotic therapeutic tablets for control release. The pharmaceutical compositions of the invention may also be in the form of oil-in-water emulsions.

Liquid preparations for oral administration may take the form of, for example, elixirs, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration can be suitably formulated to give controlled release of the active compound or prodrug, as is well known.

For buccal administration, the compositions may take the form of tablets or lozenges formulated in conventional manner.

For rectal and vaginal routes of administration, the active compound(s) can be formulated as solutions (for retention enemas) suppositories or ointments containing conventional suppository bases such as cocoa butter or other glycerides.

For nasal administration or administration by inhalation or insufflation, the active compound(s) or prodrug(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the compound and a suitable powder base such as lactose or starch.

The pharmaceutical compositions can be in the form of a sterile injectable aqueous or oleagenous suspension. This suspension can be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that can be employed are water, Ringer's solution and isotonic sodium chloride solution.

According to the invention, Syk kinase inhibitors and/or thrombopoietin receptor agonists can be used for manufacturing a composition or medicament, including medicaments suitable for rectal or urethral administration. The invention also relates to methods for manufacturing compositions including Syk kinase inhibitors and/or thrombopoietin receptor agonists in a form that is suitable for urethral or rectal administration, including suppositories.

According to the present invention, Syk kinase inhibitor and/or thrombopoietin receptor agonist compounds can also be delivered by any of a variety of inhalation devices and methods known in the art, including, for example: U.S. Pat. No. 6,241,969; U.S. Pat. No. 6,060,069; U.S. Pat. No. 6,238,647; U.S. Pat. No. 6,335,316; U.S. Pat. No. 5,364,838; U.S. Pat. No. 5,672,581; WO96/32149; WO95/24183; U.S. Pat. No. 5,654,007; U.S. Pat. No. 5,404,871; U.S. Pat. No. 5,672,581; U.S. Pat. No. 5,743,250; U.S. Pat. No. 5,419,315; U.S. Pat. No. 5,558,085; WO98/33480; U.S. Pat. No. 5,364,833; U.S. Pat. No. 5,320,094; U.S. Pat. No. 5,780,014; U.S. Pat. Nos. 5,658,878; 5,518,998; 5,506,203; U.S. Pat. No. 5,661,130; U.S. Pat. No. 5,655,523; U.S. Pat. No. 5,645,051; U.S. Pat. No. 5,622,166; U.S. Pat. No. 5,577,497; U.S. Pat. No. 5,492,112; U.S. Pat. No. 5,327,883; U.S. Pat. No. 5,277,195; U.S. Publication No. 20010041190; U.S. Publication No. 20020006901; and U.S. Publication No. 20020034477.

Included among the devices which can be used to administer particular examples of the Syk kinase inhibitor and/or thrombopoietin receptor agonist compounds are those well-known in the art, such as, metered dose inhalers, liquid nebulizers, dry powder inhalers, sprayers, thermal vaporizers, and the like. Other suitable technology for administration of particular 2,4-pyrimidinediamine compounds includes electrohydrodynamic aerosolizers.

In addition, the inhalation device is preferably practical, in the sense of being easy to use, small enough to carry conveniently, capable of providing multiple doses, and durable. Some specific examples of commercially available inhalation devices are Turbohaler (Astra, Wilmington, Del.), Rotahaler (Glaxo, Research Triangle Park, N.C.), Diskus (Glaxo, Research Triangle Park, N.C.), the Ultravent nebulizer (Mallinckrodt), the Acorn II nebulizer (Marquest Medical Products, Totowa, N.J.) the Ventolin metered dose inhaler (Glaxo, Research Triangle Park, N.C.), or the like. In one embodiment, Syk kinase inhibitor and/or thrombopoietin receptor agonist compounds can be delivered by a dry powder inhaler or a sprayer.

As those skilled in the art will recognize, the formulation of Syk kinase inhibitor and/or thrombopoietin receptor agonist compounds, the quantity of the formulation delivered, and the duration of administration of a single dose depend on the type of inhalation device employed as well as other factors. For some aerosol delivery systems, such as nebulizers, the frequency of administration and length of time for which the system is activated will depend mainly on the concentration of Syk kinase inhibitor and/or thrombopoietin receptor agonist in the aerosol. For example, shorter periods of administration can be used at higher concentrations of Syk kinase inhibitor and/or thrombopoietin receptor agonist in the nebulizer solution. Devices such as metered dose inhalers can produce higher aerosol concentrations, and can be operated for shorter periods to deliver the desired amount of Syk kinase inhibitor and/or thrombopoietin receptor agonist in some embodiments. Devices such as dry powder inhalers deliver active agent until a given charge of agent is expelled from the device. In this type of inhaler, the amount of 2 Syk kinase inhibitor and/or thrombopoietin receptor agonist in a given quantity of the powder determines the dose delivered in a single administration. The formulation of Syk kinase inhibitor and/or thrombopoietin receptor agonist is selected to yield the desired particle size in the chosen inhalation device.

Formulations of 2 Syk kinase inhibitor and/or thrombopoietin receptor agonist compounds for administration from a dry powder inhaler may typically include a finely divided dry powder containing Syk kinase inhibitor and/or thrombopoietin receptor agonist compounds, but the powder can also include a bulking agent, buffer, carrier, excipient, another additive, or the like. Additives can be included in a dry powder formulation of Syk kinase inhibitor and/or thrombopoietin receptor agonist compounds, for example, to dilute the powder as required for delivery from the particular powder inhaler, to facilitate processing of the formulation, to provide advantageous powder properties to the formulation, to facilitate dispersion of the powder from the inhalation device, to stabilize to the formulation (e.g., antioxidants or buffers), to provide taste to the formulation, or the like. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; or the like.

The present invention also relates to a pharmaceutical composition including Syk kinase inhibitor and/or thrombopoietin receptor agonist compounds suitable for administration by inhalation. According to the invention, Syk kinase inhibitor and/or thrombopoietin receptor agonist compounds can be used for manufacturing a composition or medicament, including medicaments suitable for administration by inhalation. The invention also relates to methods for manufacturing compositions including Syk kinase inhibitor and/or thrombopoietin receptor agonist compounds in a form that is suitable for administration, including administration by inhalation. For example, a dry powder formulation can be manufactured in several ways, using conventional techniques, such as described in any of the publications mentioned above and incorporated expressly herein by reference, and for example, Baker, et al., U.S. Pat. No. 5,700,904, the entire disclosure of which is incorporated expressly herein by reference. Particles in the size range appropriate for maximal deposition in the lower respiratory tract can be made by micronizing, milling, or the like. And a liquid formulation can be manufactured by dissolving the Syk kinase inhibitor and/or thrombopoietin receptor agonist compounds in a suitable solvent, such as water, at an appropriate pH, including buffers or other excipients.

Pharmaceutical compositions comprising the Syk kinase inhibitors and/or thrombopoietin receptor agonists described herein (or prodrugs thereof) can be manufactured by means of conventional mixing, dissolving, granulating, dragee-making levigating, emulsifying, encapsulating, entrapping or lyophilization processes. The compositions can be formulated in conventional manner using one or more physiologically acceptable carriers, diluents, excipients or auxiliaries which facilitate processing of the active compounds into preparations which can be used pharmaceutically.

For ocular administration, the Syk kinase inhibitor and/or thrombopoietin receptor agonist compound(s) or prodrug(s) can be formulated as a solution, emulsion, suspension, etc. suitable for administration to the eye. A variety of vehicles suitable for administering compounds to the eye are known in the art. Specific non-limiting examples are described in U.S. Pat. No. 6,261,547; U.S. Pat. No. 6,197,934; U.S. Pat. No. 6,056,950; U.S. Pat. No. 5,800,807; U.S. Pat. No. 5,776,445; U.S. Pat. No. 5,698,219; U.S. Pat. No. 5,521,222; U.S. Pat. No. 5,403,841; U.S. Pat. No. 5,077,033; U.S. Pat. No. 4,882,150; and U.S. Pat. No. 4,738,851.

For prolonged delivery, the Syk kinase inhibitor and/or thrombopoietin receptor agonist compound(s) or prodrug(s) can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the active compound(s) for percutaneous absorption can be used. To this end, permeation enhancers can be used to facilitate transdermal penetration of the active compound(s). Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems can be employed. Liposomes and emulsions are well-known examples of delivery vehicles that can be used to deliver active compound(s) or prodrug(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device can be accompanied by instructions for administration.

The Syk kinase inhibitor and/or thrombopoietin receptor agonist compound(s) or prodrug(s) described herein, or compositions thereof, will generally be used in an amount effective to achieve the intended result, for example in an amount effective to treat or prevent the particular condition being treated. The compound(s) can be administered therapeutically to achieve therapeutic benefit or prophylactically to achieve prophylactic benefit. By therapeutic benefit is meant eradication or amelioration of the underlying disorder being treated, i.e., thrombocytopenia, and/or eradication or amelioration of one or more of the symptoms associated with the underlying disorder such that the patient reports an improvement in feeling or condition, notwithstanding that the patient may still be afflicted with the underlying disorder. Therapeutic benefit also includes halting or slowing the progression of the disease, regardless of whether improvement is realized.

For prophylactic administration, the compound can be administered to a patient at risk of developing thrombocytopenia, particularly immune thrombocytopenia. Prophylactic administration can be applied to avoid the onset of symptoms in a patient diagnosed with the underlying disorder.

The amount of compound administered will depend upon a variety of factors, including, for example, the particular condition being treated, the mode of administration, the severity of the condition being treated and the age and weight of the patient, the bioavailability of the particular active compound, etc. Determination of an effective dosage is well within the capabilities of those skilled in the art.

As known by those of skill in the art, the preferred dosage of Syk kinase inhibitor and/or thrombopoietin receptor agonist compounds will also depend on the age, weight, general health and severity of the condition of the individual being treated. Dosage may also need to be tailored to the sex of the individual and/or where administered by inhalation, the lung capacity of the individual. Dosage may also be tailored to individuals suffering from more than one condition or those individuals who have additional conditions which affect lung capacity and the ability to breathe normally, for example, emphysema, bronchitis, pneumonia, respiratory infections, etc. Dosage, and frequency of administration of the compounds or prodrugs thereof, will also depend on whether the compounds are formulated for treatment of acute episodes of thrombocytopenia or for the prophylactic treatment of such a disorder. A skilled practitioner will be able to determine the optimal dose for a particular individual.

The amount of compound administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, etc. Determination of an effective dosage is well within the capabilities of those skilled in the art.

Effective dosages can be estimated initially from in vitro assays. For example, an initial dosage for use in animals can be formulated to achieve a circulating blood or serum concentration of active compound that is at or above an IC50 of the particular compound as measured in as in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, and the references cited therein.

Initial dosages can also be estimated from in vivo data, such as animal models. Animal models useful for testing the efficacy of compounds to treat or prevent the various diseases described above are well-known in the art. Suitable animal models of thrombocytopenia, such as models of immune thrombocytopenia are described in e.g., U.S. Pat. No. 6,835,809, Crow et al., Br J Haematol. 2001 December; 115(3):679-86; and Crow, et al., Blood. 2003 Jul. 15; 102(2):558-60. Epub 2003 Mar. 20. Ordinarily skilled artisans can routinely adapt such information to determine dosages suitable for human administration.

In one exemplary murine model of ITP, mice injected with an antibody directed to glycoprotein (GP)IIb (integrin α_{IIb}, CD41) become profoundly thrombocytopenic 24 hours post injection. CD-1 mice, approximately 10 weeks of age (obtainable from Charles River, Montreal, PQ, Canada) are fed rodent chow and water ad libitum. All mice, with the exception of the naïve group, are injected with 2 μg rat anti-mouse GPIIb (also referred to as anti-integrin α_{IIb} or anti-CD41) antibody (specific for GPIIb, clone MWReg30, rat IgG₁, κ, PharMingen, Missassuaga, ON, Canada) in 200 μL PBS on Day 1. Platelets are enumerated on Day 2 (24 hours following injection of anti-platelet antibody).

During the 3 day experiment (Day O-Day 2), treated mice are given experimental doses of Syk kinase inhibitor and/or thrombopoietin receptor agonist (i.e. test compound) once on Day 0, twice on Day 1 and once on Day 2. Preferably the test compound(s) is administered by oral gavage, but in some case may be administered inperitoneally or intravenously. Control animals are given an equal volume of vehicle. As a positive control, mice are injected intraperitoneally (IP) with 2 g/kg Gamunex IVIg (Bayer, Elkhart, Ill.) once on Day 0 as previously described (Crow et al., “IVIg-mediated amelioration of murine ITP via FcγRIIB is independent of SHIP1, SHP-1, and Btk activity.” Blood. 2003 Jul. 15; 102(2):558-60).

Two hours following the last treatment on Day 2, all mice are bled via the saphenous vein; 50 μL of blood is collected into 450 μL PBS/1% EDTA. The blood is further diluted to a final dilution of 1/12,000 in PBS/1% EDTA. Platelets can be enumerated by flow cytometry according to accepted methods (see e.g., Crow et al., “IVIg inhibits reticuloendothelial system function and ameliorates murine passive-immune thrombocytopenia independent of anti-idiotype reactivity.” Br J Haematol. 2001 December; 115(3):679-86.). For example, the samples can be acquired for 2 minutes on a flow-rate-calibrated FACScan flow cytometer (Becton-Dickinson, San Jose, Calif.), using forward scatter (FSC) versus side scatter (SSC) to gate platelets.

Mice injected with anti-platelet antibody are expected to become thrombocytopenic by Day 2 (one day after injection of antibody). Mean platelet counts in CD-1 mice treated with anti-platelet antibody are expected to be significantly decreased over counts in naïve (unmanipulated) mice by 24 hours following anti-GPIIb treatment (p<0.00001). Treatment of thrombocytopenic mice with inhibitors of Syk or thrombopoietin receptor agonists is expected to inhibit the reduction in platelet count by Day 2. Treatment of thrombocytopenic mice with an inhibitor of Syk and a thrombopoietin receptor agonist is anticipated to inhibit the reduction in platelet count by Day 2, to a greater extent than treatment with an inhibitor of Syk or a thrombopoietin receptor agonist alone.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration and various factors discussed above. Dosage amount and interval can be adjusted individually to provide plasma levels of the compound(s) which are sufficient to maintain therapeutic or prophylactic effect. For example, the compounds can be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

Preferably, the compound(s) will provide therapeutic or prophylactic benefit without causing substantial toxicity. Toxicity of the compound(s) can be determined using standard pharmaceutical procedures. The dose ratio between toxic and therapeutic (or prophylactic) effect is the therapeutic index. Compounds(s) that exhibit high therapeutic indices are preferred.

The foregoing disclosure pertaining to the dosage requirements for the at risk for immune thrombocytopenia compounds is pertinent to dosages required for prodrugs, with the realization, apparent to the skilled artisan, that the amount of prodrug(s) administered will also depend upon a variety of factors, including, for example, the bioavailability of the particular prodrug(s) the conversation rate and efficiency into active drug compound under the selected route of administration, etc. Determination of an effective dosage of prodrug(s) for a particular use and mode of administration is well within the capabilities of those skilled in the art.

Effective dosages can be estimated initially from in vitro activity and metabolism assays. For example, an initial dosage of prodrug for use in animals can be formulated to achieve a circulating blood or serum concentration of the metabolite active compound that is at or above an IC50 of the particular compound as measured in an in vitro assay. Calculating dosages to achieve such circulating blood or serum concentrations taking into account the bioavailability of the particular prodrug via the desired route of administration is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pagamonon Press, and the references cited therein.

Also provided are kits for administration of the Syk kinase inhibitor and thrombopoietin receptor agonist, or pharmaceutical formulations comprising the compounds, that may include a dosage amount of at least one 2,4-pyrimidinediamine or a composition comprising at least one 2,4-pyrimidinediamine as disclosed herein. Kits may further comprise suitable packaging and/or instructions for use of the compound. Kits may also comprise a means for the delivery of the at least one Syk kinase inhibitor and/or thrombopoietin receptor agonist or compositions comprising at least one Syk kinase inhibitor and/or thrombopoietin receptor agonist, such as an inhaler, spray dispenser (e.g. nasal spray), syringe for injection or pressure pack for capsules, tables, suppositories, or other device as described herein.

Additionally, the compounds of the present invention can be assembled in the form of kits. The kit provides the compound and reagents to prepare a composition for administration. The composition can be in a dry or lyophilized form, or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to syringe, pipette, transdermal patch, or inhalant.

The kits will include appropriate instructions for preparation and administration of the composition, side effects of the compositions, and any other relevant information. The instructions can be in any suitable format, including, but not limited to, printed matter, videotape, computer readable disk, or optical disc.

In another embodiment, this invention provides a kit comprising the pharmaceutical formulation comprising a Syk kinase inhibitor and/or thrombopoietin receptor agonist, as described herein, or a prodrug thereof, and at least one pharmaceutically acceptable excipient, diluent, preservative, or stabilizer, or mixtures thereof, packaging, and instructions for use.

In another aspect of the invention, kits for treating an individual who suffers from or is susceptible to the conditions described herein are provided, comprising a container comprising a dosage amount of a Syk kinase inhibitor and/or thrombopoietin receptor agonist or composition as disclosed herein, and instructions for use. The container can be any of those known in the art and appropriate for storage and delivery of oral, intravenous, topical, rectal, urethral, or inhaled formulations.

Kits may also be provided that contain sufficient dosages of the Syk kinase inhibitor and/or thrombopoietin receptor agonist or composition to provide effective treatment for an individual for an extended period, such as a week, 2 weeks, 3, weeks, 4 weeks, 6 weeks or 8 weeks or more.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Methods:

### Example 1

On Day 1 of each study, mice were rendered thrombocytopenic by injection with rat anti-mouse CD41 antibody (IgG1, Clone MWReg30) by the intraperitoneal (IP) route. The platelet depleting antibody (rat anti-mouse GPIIb, also referred to as anti-integrin α_{IIb} or anti-CD41) was administered prior to treatment with Compound I on Day 1. Mice in control groups either received nothing (naïve), saline only, or an isotype-matched irrelevant control antibody (nonspecific rat anti-mouse IgG1, Clone R3-34, BD, San Jose, Calif.).

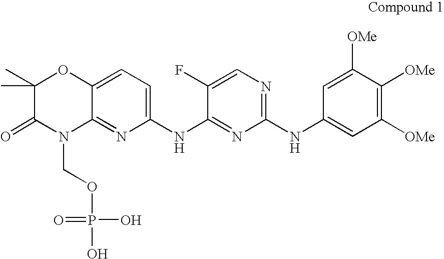

Mice received vehicle or Compound 1 at various dose levels (0, 20, 40, or 80 mg/kg, PO, 5 mL/kg) 30 minutes prior to the platelet depleting antibody, then again 8 h, 16 h, and 23 h after induction of thrombocytopenia. At 24 h after administration of the anti-CD41 platelet-depleting antibody, blood was harvested from all groups by cardiocentesis to assess the platelet counts or drug levels in the plasma. An aliquot from each sample was transferred to a plasma separator tube and the separated plasma was stored at −80° C. for analysis. Blood was placed indirectly on ice for delivery. Platelet enumeration was performed using Bayer Advia 120 Bayer Advia 120 hematology system (Bayer, Tarrytown, N.Y.) by Quality Clinical Laboratories (QCL, Mountain View, Calif.).

Systemic drug exposures were determined at sacrifice with the final dose being given on the evening of Day 1 (8 h prior to blood collection) or on the morning of Day 2 (1 h prior to blood collection). Satellite animals treated with Compound 1 and not receiving platelet-depleting antibody were added to obtain a more complete PK profile.

Significant differences between vehicle and Compound I-treated groups were determined statistically using the GraphPad Prism 4.0® software package, unless otherwise stated. In general, Prism 4.0® uses the standard calculations as detailed in D. G. Altman, Practical Statistics for Medical Research, 1991, Chapman and Hall. Platelet count data was analyzed using Kruskal Wallis Nonparametric test with Dunn's multiple group post-hoc test to compare the rank sums, unless otherwise noted.

### Example 2

In vivo studies showed reduced depletion of platelets in CD-1 outbred mice with Compound 1 treatment prior to and following administration of CD41 platelet depleting antibody.

CD-1 mice, approximately 10 weeks of age, were obtained from Charles River (Montreal, PQ, Canada). All mice, with the exception of the naïve group, were injected with 2 μg rat anti-mouse GPIIb (also referred to as anti-integrin α_{IIb} or anti-CD41) antibody (specific for GPIIb, clone MWReg30, rat IgG₁, κ, PharMingen, Missassuaga, ON, Canada) in 200 μL PBS on Day 1. Platelets were enumerated on Day 2 (24 hours following injection of anti-platelet antibody).

During the 3 day experiment (Day O— Day 2), Compound I-treated mice were given the indicated doses of Compound 1 by oral gavage once on Day 0, twice on Day 1 and once on Day 2. Control animals were given an equal volume of vehicle. For the IVIg group, mice were injected intraperitoneally (IP) with 2 g/kg Gamunex IVIg (Bayer, Elkhart, Ill.) once on Day 0. Two concentrations of Compound 1 (3.08 and 4.94 mg/mL) were formulated with 0.1% CMC Na-0.1% Methylparaben-0.02% Propylparaben-H₂O. One day prior to platelet depletion, treatment began. Mice were treated orally twice daily (bid.) with Compound 1 (30.8 or 49.4 mg/kg of body weight per dose, n=6) at a dose volume of 10 mL/kg. IVIg (2 g/kg) was used as a reference agent and was administered IP as described previously (Crow et al., 2003).

Two hours following the last treatment of Compound 1 on Day 2, all mice were bled via the saphenous vein; 50 μL of blood was collected into 450 μL PBS/1% EDTA. The blood was further diluted to a final dilution of 1/12,000 in PBS/1% EDTA. Platelets were enumerated by flow cytometry according to Crow et al., 2001. Briefly, the samples were acquired for 2 minutes on a flow-rate-calibrated FACScan flow cytometer (Becton-Dickinson, San Jose, Calif.), using forward scatter (FSC) versus side scatter (SSC) to gate platelets.

All values in the text and figures are expressed as mean±standard error of the mean (SEM) for n=6 mice/group unless noted otherwise, and n represents the number of mice. Differences between treatment and control groups were compared using the 2-tailed Student's t-test using GraphPad Prism Inc., software ver. 4.0 (San Diego, Calif., USA).

### Results:

### Example 3

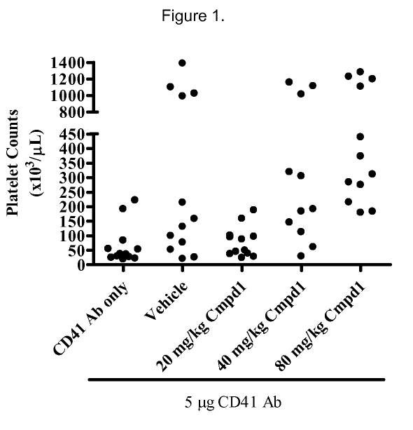

Animals treated with 40 mg/kg or 80 mg/kg Compound 130 minutes prior to administration of platelet-depleting antibody demonstrated a significantly higher platelet count than mice treated with CD41 antibody alone (FIG. 1, p<0.05 and p<0.001, respectively). Moreover, platelet counts in animals given platelet depleting antibody and 20 mg/kg Compound 1 were significantly lower than counts from the 80 mg/kg group (p<0.01), suggesting a dose-dependent inhibition of platelet depletion with Compound 1 treatment.

FIG. 1 shows the effect of Compound 1 treatment on platelet counts in c57bl/6 mice. In FIG. 1, each data point represents platelet counts for an individual mouse administered a single IP injection of 5 μg of CD41 platelet depleting antibody (n=11-12 per group). Mice were bled 24 hours post antibody administration and platelet counts were performed using Bayer Advia 120 analyzer. Mice were given vehicle or indicated doses of Compound 1. Platelet-depleting antibody was administered 30 minutes after the first Compound 1 or vehicle dose on Day 1.

### Example 4

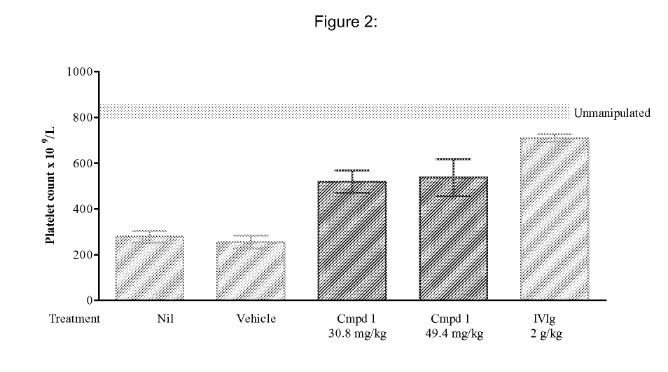

Mice injected with anti-platelet antibody became thrombocytopenic by Day 2 (one day after injection of antibody). Mean platelet counts in CD-1 mice treated with anti-platelet antibody were significantly decreased over counts in naïve (unmanipulated) mice by 24 hours following anti-GPIIb treatment (p<0.00001). Treatment of thrombocytopenic mice with Compound 1 throughout the experiment inhibited the reduction in platelet count by Day 2. Inhibition of platelet depletion following treatment with Compound 1 was highly significant compared with animals treated with vehicle alone (p=0.001 for the 30.8 mg/kg group and p=0.008 for the 49.4 mg/kg group) (FIG. 2). The effect of Compound 1 in this study did not appear to be dose-dependent, as similar results were seen at 30.8 and 49.4 mg/kg. The difference in platelet counts between the two treatment groups was not significant (p=0.9). Platelet counts in animals treated with vehicle alone were similar to animals treated with anti-GPIIb antibody (p=0.55). Treatment of mice with 2 g/kg IVIg successfully prevented the disease in CD-1, and mean platelets counts were similar to naïve mice (p=0.018). Table 1 shows platelet counts 24 hours post antibody treatment.

FIG. 2 shows the effect of Compound 1 on antibody-mediated thrombocytopenia. On Day 0, mice were pre-treated with nothing (Nil), 0.25 mL Vehicle twice (8 hrs apart) or the indicated doses of Compound 1 (in 0.25 mL vehicle) or 2 g/kg IVIg. On Day 1, the above treatments were repeated except for IVIg. All mice also received 2 μg anti-platelet antibody on Day 1. On Day 2, all mice were bled for platelet enumeration by flow cytometry as described. n=6 mice per group. Data are expressed as mean±SEM.

TABLE 1
Platelet Counts 24 Hours Following Anti-α_{IIb} Treatment

| Group | Individual Animal Platelet Count × 10⁹/L | | | | | | Mean | SEM |
|---|---|---|---|---|---|---|---|---|
| Naïve | 734 | 672 | 797 | 786 | 642 | 876 | 751 | 35 |
| Antibody | 210 | 327 | 197 | 277 | 340 | 321 | 279 | 25 |
| Vehicle | 198 | 202 | 209 | 329 | 240 | 354 | 255 | 28 |
| Compound 1 | 392 | 398 | 457 | 584 | 602 | 679 | 519 | 49 |
| 30.8 mg/kg | | | | | | | | |
| Compound 1 | 411 | 437 | 433 | 498 | 931 | 510 | 537 | 80 |
| 49.4 mg/kg | | | | | | | | |
| 2g/kg IVIg | 700 | 742 | 755 | 732 | 680 | 650 | 710 | 16 |

What is claimed is:

1. A method of increasing platelet levels in a patient having or at risk for immune thrombocytopenia comprising co-administering:
(a) a Syk kinase inhibitor; and
(b) a thrombopoietin receptor agonist.

2. The method of claim 1, wherein the Syk kinase inhibitor is a 2,4-pyrimidinediamine compound of formula I: 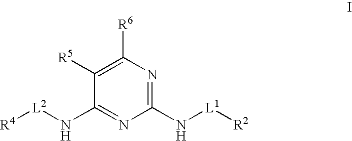
a salt, hydrate, solvate, N-oxide or prodrug thereof, wherein:
L¹ and L², independently of each other, are selected from the group consisting of a direct bond, a (C1-C3) alkylene optionally substituted with one or more of the same or different R⁹ groups, and 1-3 membered heteroalkyldiyl optionally substituted with one or more of the same or different R⁹ groups;
R² is selected from the group consisting of (C1-C6) alkyl optionally substituted with one or more of the same or different R⁸ groups, (C2-C6) alkenyl optionally substituted with one or more of the same or different R⁸ groups, (C2-C6) alkynyl optionally substituted with one or more of the same or different R⁸ groups, (C3-C8) cycloalkyl optionally substituted with one or more of the same or different R⁸ groups, 3-8 membered heterocyclyl optionally substituted with one or more of the same or different R⁸ groups, (C5-C15) aryl optionally substituted with one or more of the same or different R⁸ groups, and 5-15 membered heteroaryl optionally substituted with one or more of the same or different R⁸ groups;
R⁴ is selected from the group consisting of hydrogen, (C1-C6) alkyl optionally substituted with one or more of the same or different R⁸ groups, (C2-C6) alkenyl optionally substituted with one or more of the same or different R⁸ groups, (C2-C6) alkynyl optionally substituted with one or more of the same or different R⁸ groups, (C3-C8) cycloalkyl optionally substituted with one or more of the same or different R⁸ groups, 3-8 membered heterocyclyl optionally substituted with one or more of the same or different R⁸ groups, (C5-C15) aryl optionally substituted with one or more of the same or different R⁸ groups, and 5-15 membered heteroaryl optionally substituted with one or more of the same or different R⁸ groups;
R⁵ is selected from the group consisting of R⁶, (C2-C6) alkenyl optionally substituted with one or more of the same or different R⁸ groups, (C2-C6) alkynyl optionally substituted with one or more of the same or different R⁸ groups, and (C3-C8) cycloalkyl optionally substituted with one or more of the same or different R⁸ groups;
each R⁶, independently of the others, is selected from the group consisting of hydrogen, —OR^{d}, —SR^{d}, (C1-C3) haloalkyloxy, (C1-C3) perhaloalkyloxy, —NR^{c}R^{c}, halogen, (C1-C3) haloalkyl, (C1-C3) perhaloalkyl, —CN, —NC, —OCN, —SCN, —NO, —NO₂, —N₃, —S(O)R^{d}, —S(O)₂R^{d}, —S(O)NR^{c}R^{c}, —S(O)₂NR^{c}R^{c}, —OS(O) R^{d}, —OS(O)₂Rd, —OS(O)₂OR^{d}, —OS(O)NR^{c}R^{c}, —OS(O)₂NR^{c}R^{c}, —C(O)R^{d}, —C(O)OR^{d}, —C(O)NR^{c}R^{c}, —C(NH)NR^{c}R^{c}, —OC(O)R^{d}, —SC(O)R^{d}, —OC(O)OR^{d}, —SC(O)OR^{d}, —OC(O)NR^{c}R^{c}, —SC(O)NR^{c}R^{c}, —OC(NH)NR^{c}R^{c}, —SC(NH)NR^{c}R^{c}, —[NHC(O)]ₙR^{d}, —[NHC(O)]ₙOR^{d}, —[NHC(O)]ₙR^{c}R^{c}, —[NHC(NH)]ₙR^{c}R^{c}, (C5-C10) aryl optionally substituted with one or more of the same or different R⁸ groups, (C6-C16) arylalkyl optionally substituted with one or more of the same or different R⁸ groups, 5-10 membered heteroaryl optionally substituted with one or more of the same or different R⁸ groups, and 6-16 membered heteroarylalkyl optionally substituted with one or more of the same or different R⁸ groups;
R⁸ is selected from the group consisting of R^{e}, R^{b}, R^{e} substituted with one or more of the same or different R^{a} or R^{b}, —OR^{a} substituted with one or more of the same or different R^{a} or R^{b}, —B(OR^{a})₂, —B(NR^{c}R^{c})₂, —(CH₂)ₘ—R^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂, —O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b}, —C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b}, —NH—(CHR^{a})ₘ—R^{b}, —N[(CH₂)ₘR^{b}]₂, —NH—C(O)—NH—(CH₂)ₘ—R^{b}, —NH—C(O)—(CH₂)ₘ—CHR^{b}R^{b} and —NH—(CH₂)ₘ—C(O)—NH—(CH₂)ₘ—R^{b};
each R⁹, independently of the others, is selected from the group consisting of (C1-C6) alkyl, —OR^{a}, —C(O)OR^{a}, (C5-C10) aryl optionally substituted with one or more of the same or different halogens, phenyl optionally substituted with one or more of the same or different halogens, and 5-10 membered heteroaryl optionally substituted with one or more of the same or different halogens;
each R^{a}, independently of the others, is selected from the group consisting of hydrogen, (C1-C6) alkyl, (C3-C8) cycloalkyl, (C4-C11) cycloalkylalkyl, (C5-C10) aryl, (C6-C16) arylalkyl, 2-6 membered heteroalkyl, 3-8 membered heterocyclyl, 4-11 membered heterocyclylalkyl, 5-10 membered heteroaryl and 6-16 membered heteroarylalkyl;
each R^{b}, independently of the others, is a suitable group selected from the group consisting of ═O, —OR^{d}, (C1-C3) haloalkyloxy, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, S(O)₂OR^{c}, S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c}, independently of the others, is a progroup or R^{a}, or, alternatively, two R^{c} are taken together with the nitrogen atom to which they are bonded to form a 5 to 8-membered heterocyclyl or heteroaryl which may optionally include one or more of the same or different additional heteroatoms and which may optionally be substituted with one or more of the same or different R^{a} or suitable R^{b} groups;
each R^{d}, independently of the others, is a progroup or R^{a};
each R^{e}, independently of the others, is selected from the group consisting of (C1-C6) alkyl, (C3-C8) cycloalkyl, (C4-C11) cycloalkylalkyl, (C5-C10) aryl, (C6-C16) arylalkyl, 2-6 membered heteroalkyl, 3-8 membered heterocyclyl, 4-11 membered cycloheteroalkylalkyl, 5-10 membered heteroaryl and 6-16 membered heteroarylalkyl;
each m, independently of the others, is an integer from 1 to 3; and
each n, independently of the others, is an integer from 0 to 3.

3. The method of claim 1, wherein the Syk kinase inhibitor is a compound of formula II 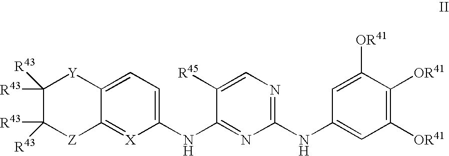
or a pharmaceutically acceptable salt or N-oxide thereof, wherein
X is selected from the group consisting of N and CH;
Y is selected from the group consisting of O, S, SO, SO₂, SONR⁴¹, NH, and NR⁴²;
Z is selected from the group consisting of O, S, SO, SO₂, SONR⁴¹, NH, and NR⁴²;
each R⁴¹, independently of the others, is hydrogen or lower alkyl;
each R⁴², independently of the others, is selected from the group consisting of alkyl, substituted alkyl, alkenyl, substituted alkenyl, alkynyl, substituted alkynyl, cycloalkynyl, substituted cycloalkynyl, aryl, substituted aryl, cycloalkyl, substituted cycloalkyl, heteroaryl, substituted heteroaryl, heterocyclyl, substituted heterocyclyl, hydroxyl, alkoxy, substituted alkoxy, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxyl ester, (carboxyl ester)oxy, and —(CRR)ₙ—R^{p}, wherein
each R, independently of the others, is selected from the group consisting of hydrogen, lower alkyl and halo;
n is an integer from 0 to 4; and
R^{p} is selected from the group consisting of phosphate, phosphate ester, phosphonate, phosphorodiamidate, phosphoramidate monoester, phosphoramidate diester, cyclic phosphoramidate, cyclic phosphorodiamidate, phosphonamidate, and cyclic phosphonamidate;
each R⁴³, independently of the others, is selected from the group consisting of hydrogen, alkyl, substituted alkyl, alkoxy, substituted alkoxy, alkenyl, substituted alkenyl, alkynyl, substituted alkynyl, cycloalkynyl, substituted cycloalkynyl, alkynyloxy, amino, substituted amino, aryl, substituted aryl, aryloxy, substituted aryloxy, cyano, cycloalkyl, substituted cycloalkyl, cycloalkoxy, substituted cycloalkoxy, heteroaryl, substituted heteroaryl, heteroaryloxy, substituted heteroaryloxy, heterocyclyl, substituted heterocyclyl, heterocyclyloxy, substituted heterocyclyloxy, aminocarbonyl, aminocarbonyloxy, carboxyl, carboxyl ester, (carboxyl ester)oxy, nitro, and halo, or, alternatively, two R⁴³ bonded to the same carbon atom are taken together to form an oxo (═O), ═NH or ═NR⁴⁴ group and the other two R⁴³ are as defined above;
each R⁴⁴, independently of the others, is selected from the group consisting of (C1-C6) alkyl and (C5-C14) aryl; and
R⁴⁵ is selected from the group consisting of alkyl, substituted alkyl, alkoxy, substituted alkoxy, amino, substituted amino, carboxyl, carboxyl ester, cyano, halo, nitro, alkenyl, substituted alkenyl, alkynyl and substituted alkynyl.

4. The method of claim 3, wherein two R⁴³ bonded to the same carbon atom are taken together to form an oxo (═O) group and the other two R⁴³ independently of one another are hydrogen or alkyl.

5. The method of claim 3, wherein X is N.

6. The method of claim 3, wherein each R⁴¹ is methyl.

7. The method of claim 3, wherein Y is O and Z is NH or NR⁴².

8. The method of claim 7, wherein Z is NR⁴² and R⁴² is —(CRR)ₙ—R^{p}.

9. The method of claim 8, wherein n is one.

10. The method of claim 9, wherein each R is hydrogen and R^{p} is phosphate or phosphate ester.

11. The method of claim 1, wherein the thrombopoietin receptor agonist is a polypeptide.

12. The method of claim 11, wherein the polypeptide is thrombopoietin.

13. The method of claim 11, wherein the polypeptide comprises the sequence Ile-Glu-Gly-Pro-Thr-Leu-Arg-Gln-Trp-Leu-Ala-Ala-Arg-Ala (SEQ ID NO: 1).

14. The method of claim 11, wherein the polypeptide is AMG531.

15. The method of claim 1, wherein the thrombopoietin receptor agonist is a small molecule.

16. The method of claim 15, wherein the small molecule is 3′-{N′-[1-(3,4-dimethylphenyl)-3-methyl-5-oxo-1,5-di-hydropyrazol-4-ylidene]hydrazine}-2′-hydroxybiphenyl-3-carboxylic acid, or a pharmaceutically acceptable salt or ester thereof.

17. The method of claim 15, wherein the small molecule is eltrombopag.

18. The method of claim 1, wherein the immune thrombocytopenia is immune thrombocytopenia purpura.

19. A method of treating thrombocytopenia comprising co-administering to a patient in need thereof a Syk kinase inhibitor and a thrombopoietin receptor agonist.

20. The method of claim 19, wherein the Syk kinase inhibitor is a 2,4, pyrimidinediamine compound of formula I: 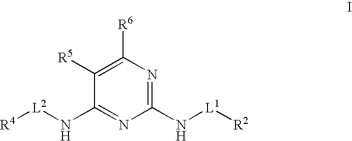
a salt, hydrate, solvate, N-oxide or prodrug thereof, wherein:
L¹ and L², independently of each other, are selected from the group consisting of a direct bond, a (C1-C3) alkylene optionally substituted with one or more of the same or different R⁹ groups, and 1-3 membered heteroalkyldiyl optionally substituted with one or more of the same or different R⁹ groups;
R² is selected from the group consisting of (C1-C6) alkyl optionally substituted with one or more of the same or different R⁸ groups, (C2-C6) alkenyl optionally substituted with one or more of the same or different R⁸ groups, (C2-C6) alkynyl optionally substituted with one or more of the same or different R⁸ groups, (C3-C8) cycloalkyl optionally substituted with one or more of the same or different R⁸ groups, 3-8 membered heterocyclyl optionally substituted with one or more of the same or different R⁸ groups, (C5-C15) aryl optionally substituted with one or more of the same or different R⁸ groups, and 5-15 membered heteroaryl optionally substituted with one or more of the same or different R⁸ groups;
R⁴ is selected from the group consisting of hydrogen, (C1-C6) alkyl optionally substituted with one or more of the same or different R⁸ groups, (C2-C6) alkenyl optionally substituted with one or more of the same or different R⁸ groups, (C2-C6) alkynyl optionally substituted with one or more of the same or different R⁸ groups, (C3-C8) cycloalkyl optionally substituted with one or more of the same or different R⁸ groups, 3-8 membered heterocyclyl optionally substituted with one or more of the same or different R⁸ groups, (C5-C15) aryl optionally substituted with one or more of the same or different R⁸ groups, and 5-15 membered heteroaryl optionally substituted with one or more of the same or different R⁸ groups;
R⁵ is selected from the group consisting of R⁶, (C2-C6) alkenyl optionally substituted with one or more of the same or different R⁸ groups, (C2-C6) alkynyl optionally substituted with one or more of the same or different R⁸ groups, and (C3-C8) cycloalkyl optionally substituted with one or more of the same or different R⁸ groups;
each R⁶, independently of the others, is selected from the group consisting of hydrogen, —OR^{d}, —SR^{d}, (C1-C3) haloalkyloxy, (C1-C3) perhaloalkyloxy, —NR^{c}R^{c}, halogen, (C1-C3) haloalkyl, (C1-C3) perhaloalkyl, —CN, —NC, —OCN, —SCN, —NO, —NO₂, —N₃, —S(O)R^{d}, —S(O)₂R^{d}, —S(O)NR^{c}R^{c}, —S(O)₂NR^{c}R^{c}, —OS(O)R^{d}, —OS(O)₂Rd, —OS(O)₂R^{d}, —OS(O)NR^{c}R^{c}, —OS(O)₂NR^{c}R^{c}, —C(O)R^{d}, —C(O)OR^{d}, —C(O)NR^{c}R^{c}, —C(NH)NR^{c}R^{c}, —OC(O)R^{d}, —SC(O)R^{d}, —OC(O)OR^{d}, —SC(O)OR^{d}, —OC(O)NR^{c}R^{c}, —SC(O)NR^{c}R^{c}, —OC(NH)NR^{c}R^{c}, —SC(NH)NR^{c}R^{c}, —[NHC(O)]ₙR^{d}, —[NHC(O)]ₙOR^{d}, —[NHC(O)]ₙR^{c}R^{c}, —[NHC(NH)]ₙR^{c}R^{c}, (C5-C10) aryl optionally substituted with one or more of the same or different R⁸ groups, (C6-C16) arylalkyl optionally substituted with one or more of the same or different R⁸ groups, 5-10 membered heteroaryl optionally substituted with one or more of the same or different R⁸ groups, and 6-16 membered heteroarylalkyl optionally substituted with one or more of the same or different R⁸ groups;
R⁸ is selected from the group consisting of R^{e}, R^{b}, R^{e} substituted with one or more of the same or different R^{a} or R^{b}, —OR^{a} substituted with one or more of the same or different R^{a} or R^{b}, —B(OR^{a})₂, —B(NR^{c}R^{c})₂, —(CH₂)ₘR^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂, —O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b}, —C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b}, —NH—(CHR^{a})ₘ—R^{b}, —N[(CH₂)ₘR^{b}]₂, —NH—C(O)—NH—(CH₂)ₘ—R^{b}, —NH—C(O)—(CH₂)ₘ—CHR^{b}R^{b} and —NH—(CH₂)ₘ—C(O)—NH—(CH₂)ₘ—R^{b};
each R⁹, independently of the others, is selected from the group consisting of (C1-C6) alkyl, —OR^{a}, —C(O)OR^{a}, (C5-C10) aryl optionally substituted with one or more of the same or different halogens, phenyl optionally substituted with one or more of the same or different halogens, and 5-10 membered heteroaryl optionally substituted with one or more of the same or different halogens;
each R^{a}, independently of the others, is selected from the group consisting of hydrogen, (C1-C6) alkyl, (C3-C8) cycloalkyl, (C4-C11) cycloalkylalkyl, (C5-C10) aryl, (C6-C16) arylalkyl, 2-6 membered heteroalkyl, 3-8 membered heterocyclyl, 4-11 membered heterocyclylalkyl, 5-10 membered heteroaryl and 6-16 membered heteroarylalkyl;
each R^{b}, independently of the others, is a suitable group selected from the group consisting of ═O, —OR^{d}, (C1-C3) haloalkyloxy, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, S(O)₂OR^{c}, S(O)NR^{c}R^{c}, —S(O)₂NR^{c}R^{c}, —OS(O)R^{d}, —OS(O)₂OR^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c}, independently of the others, is a progroup or R^{a}, or, alternatively, two R^{c} are taken together with the nitrogen atom to which they are bonded to form a 5 to 8-membered heterocyclyl or heteroaryl which may optionally include one or more of the same or different additional heteroatoms and which may optionally be substituted with one or more of the same or different R^{a} or suitable R^{b} groups;
each R^{d}, independently of the others, is a progroup or R^{a};
each R^{e}, independently of the others, is selected from the group consisting of (C1-C6) alkyl, (C3-C8) cycloalkyl, (C4-C11) cycloalkylalkyl, (C5-C10) aryl, (C6-C16) arylalkyl, 2-6 membered heteroalkyl, 3-8 membered heterocyclyl, 4-11 membered cycloheteroalkylalkyl, 5-10 membered heteroaryl and 6-16 membered heteroarylalkyl;
each m, independently of the others, is an integer from 1 to 3; and
each n, independently of the others, is an integer from 0 to 3.

21. The method of claim 19, wherein the Syk kinase inhibitor is a 2,4-pyrimidinediamine compound of formula II 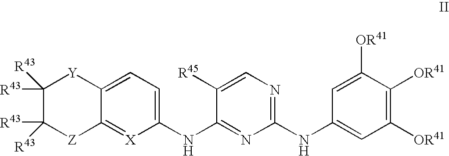
or a pharmaceutically acceptable salt or N-oxide thereof, wherein
X is selected from the group consisting of N and CH;
Y is selected from the group consisting of O, S, SO, SO₂, SONR⁴¹, NH, and NR⁴²;
Z is selected from the group consisting of O, S, SO, SO₂, SONR⁴¹, NH, and NR⁴²;
each R⁴¹, independently of the others, is hydrogen or lower alkyl;
each R⁴², independently of the others, is selected from the group consisting of alkyl, substituted alkyl, alkenyl, substituted alkenyl, alkynyl, substituted alkynyl, cycloalkynyl, substituted cycloalkynyl, aryl, substituted aryl, cycloalkyl, substituted cycloalkyl, heteroaryl, substituted heteroaryl, heterocyclyl, substituted heterocyclyl, hydroxyl, alkoxy, substituted alkoxy, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxyl ester, (carboxyl ester)oxy, and —(CRR)ₙ—R^{p}, wherein
each R^{p}, independently of the others, is selected from the group consisting of hydrogen, lower alkyl and halo;
n is an integer from 0 to 4; and
R^{p} is selected from the group consisting of phosphate, phosphate ester, phosphonate, phosphorodiamidate, phosphoramidate monoester, phosphoramidate diester, cyclic phosphoramidate, cyclic phosphorodiamidate, phosphonamidate, and cyclic phosphonamidate;
each R⁴³, independently of the others, is selected from the group consisting of hydrogen, alkyl, substituted alkyl, alkoxy, substituted alkoxy, alkenyl, substituted alkenyl, alkynyl, substituted alkynyl, cycloalkynyl, substituted cycloalkynyl, alkynyloxy, amino, substituted amino, aryl, substituted aryl, aryloxy, substituted aryloxy, cyano, cycloalkyl, substituted cycloalkyl, cycloalkoxy, substituted cycloalkoxy, heteroaryl, substituted heteroaryl, heteroaryloxy, substituted heteroaryloxy, heterocyclyl, substituted heterocyclyl, heterocyclyloxy, substituted heterocyclyloxy, aminocarbonyl, aminocarbonyloxy, carboxyl, carboxyl ester, (carboxyl ester)oxy, nitro, and halo, or, alternatively, two R⁴³ bonded to the same carbon atom are taken together to form an oxo (═O), ═NH or ═NR⁴⁴ group and the other two R⁴³ are as defined above;
each R⁴⁴, independently of the others, is selected from the group consisting of (C1-C6) alkyl and (C5-C14) aryl; and
R⁴⁵ is selected from the group consisting of alkyl, substituted alkyl, alkoxy, substituted alkoxy, amino, substituted amino, carboxyl, carboxyl ester, cyano, halo, nitro, alkenyl, substituted alkenyl, alkynyl and substituted alkynyl.

22. A pharmaceutical composition comprising a Syk kinase inhibitor and a thrombopoietin receptor agonist.

23. The pharmaceutical composition of claim 22, wherein the Syk kinase inhibitor is a 2,4-pyrimidinediamine compound of formula I 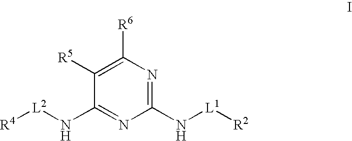
a salt, hydrate, solvate, N-oxide or prodrug thereof, wherein:
L¹ and L², independently of each other, are selected from the group consisting of a direct bond, a (C1-C3) alkylene optionally substituted with one or more of the same or different R⁹ groups, and 1-3 membered heteroalkyldiyl optionally substituted with one or more of the same or different R⁹ groups;
R² is selected from the group consisting of (C1-C6) alkyl optionally substituted with one or more of the same or different R⁸ groups, (C2-C6) alkenyl optionally substituted with one or more of the same or different R⁸ groups, (C2-C6) alkynyl optionally substituted with one or more of the same or different R⁸ groups, (C3-C8) cycloalkyl optionally substituted with one or more of the same or different R⁸ groups, 3-8 membered heterocyclyl optionally substituted with one or more of the same or different R⁸ groups, (C5-C15) aryl optionally substituted with one or more of the same or different R⁸ groups, and 5-15 membered heteroaryl optionally substituted with one or more of the same or different R⁸ groups;
R⁴ is selected from the group consisting of hydrogen, (C1-C6) alkyl optionally substituted with one or more of the same or different R⁸ groups, (C2-C6) alkenyl optionally substituted with one or more of the same or different R⁸ groups, (C2-C6) alkynyl optionally substituted with one or more of the same or different R⁸ groups, (C3-C8) cycloalkyl optionally substituted with one or more of the same or different R⁸ groups, 3-8 membered heterocyclyl optionally substituted with one or more of the same or different R⁸ groups, (C5-C15) aryl optionally substituted with one or more of the same or different R⁸ groups, and 5-15 membered heteroaryl optionally substituted with one or more of the same or different R⁸ groups;
R⁵ is selected from the group consisting of R⁶, (C2-C6) alkenyl optionally substituted with one or more of the same or different R⁸ groups, (C2-C6) alkynyl optionally substituted with one or more of the same or different R⁸ groups, and (C3-C8) cycloalkyl optionally substituted with one or more of the same or different R⁸ groups;
each R⁶, independently of the others, is selected from the group consisting of hydrogen, —OR^{d}, —SR^{d}, (C1-C3) haloalkyloxy, (C1-C3) perhaloalkyloxy, —NR^{c}R^{c}, halogen, (C1-C3) haloalkyl, (C1-C3) perhaloalkyl, —CN, —NC, —OCN, —SCN, —NO, —NO₂, —N₃, —S(O)R^{d}, —S(O)₂R^{d}, —S(O)NR^{c}R^{c}, —S(O)₂NR^{c}R^{c}, —OS(O)R^{d}, —OS(O)₂R^{d}, —OS(O)₂OR^{d}, —OS(O)NR^{c}R^{c}, —OS(O)₂NR^{c}R^{c}, —C(O)R^{d}, —C(O)OR^{d}, —C(O)NR^{c}R^{c}, —C(NH)NR^{c}R^{c}, —OC(O)R^{d}, —SC(O)R^{d}, —OC(O)OR^{d}, —SC(O)OR^{d}, —OC(O)NR^{c}R^{c}, —SC(O)NR^{c}R^{c}, —OC(NH)NR^{c}R^{c}, —SC(NH)NR^{c}R^{c}, —[NHC(O)]ₙR^{d}, —[NHC(O)]ₙOR^{d}, —[NHC(O)]ₙR^{c}R^{c}, —[NHC(NH)]ₙR^{c}R^{c}, (C5-C10) aryl optionally substituted with one or more of the same or different R⁸ groups, (C6-C16) arylalkyl optionally substituted with one or more of the same or different R⁸ groups, 5-10 membered heteroaryl optionally substituted with one or more of the same or different R⁸ groups, and 6-16 membered heteroarylalkyl optionally substituted with one or more of the same or different R⁸ groups;
R⁸ is selected from the group consisting of R^{e}, R^{b}, R^{e} substituted with one or more of the same or different R^{a} or R^{b}, —OR^{a} substituted with one or more of the same or different R^{a} or R^{b}, —B(OR^{a})₂, —B(NR^{c}R^{c})₂, —(CH₂)ₘ—R^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂, —O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b}, —C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b}, —NH—(CHR^{a})ₘ—R^{b}, —N[(CH₂)ₘR^{b}]₂, —NH—C(O)—NH—(CH₂)ₘ—R^{b}, —NH—C(O)—(CH₂)ₘ—CHR^{b}R^{b} and —NH—(CH₂)ₘ—C(O)—NH—(CH₂)ₘ—R^{b};
each R⁹, independently of the others, is selected from the group consisting of (C1-C6) alkyl, —OR^{a}, —C(O)OR^{a}, (C5-C10) aryl optionally substituted with one or more of the same or different halogens, phenyl optionally substituted with one or more of the same or different halogens, and 5-10 membered heteroaryl optionally substituted with one or more of the same or different halogens;
each R^{a}, independently of the others, is selected from the group consisting of hydrogen, (C1-C6) alkyl, (C3-C8) cycloalkyl, (C4-C11) cycloalkylalkyl, (C5-C10) aryl, (C6-C16) arylalkyl, 2-6 membered heteroalkyl, 3-8 membered heterocyclyl, 4-11 membered heterocyclylalkyl, 5-10 membered heteroaryl and 6-16 membered heteroarylalkyl;
each R^{b}, independently of the others, is a suitable group selected from the group consisting of ═O, —OR^{d}, (C1-C3) haloalkyloxy, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{c}, S(O)NR^{c}R^{c}, S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c}, independently of the others, is a progroup or R^{a}, or, alternatively, two R^{c} are taken together with the nitrogen atom to which they are bonded to form a 5 to 8-membered heterocyclyl or heteroaryl which may optionally include one or more of the same or different additional heteroatoms and which may optionally be substituted with one or more of the same or different R^{a} or suitable R^{b} groups;
each R^{d}, independently of the others, is a progroup or R^{a};
each R^{e}, independently of the others, is selected from the group consisting of (C1-C6) alkyl, (C3-C8) cycloalkyl, (C4-C11) cycloalkylalkyl, (C5-C10) aryl, (C6-C16) arylalkyl, 2-6 membered heteroalkyl, 3-8 membered heterocyclyl, 4-11 membered cycloheteroalkylalkyl, 5-10 membered heteroaryl and 6-16 membered heteroarylalkyl;
each m, independently of the others, is an integer from 1 to 3; and
each n, independently of the others, is an integer from 0 to 3.

24. The pharmaceutical composition of claim 22, wherein the Syk kinase inhibitor is a 2,4-pyrimidinediamine compound of formula II 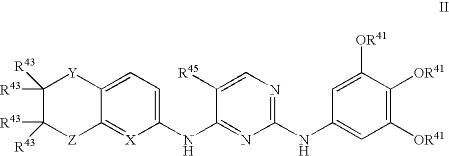
or a pharmaceutically acceptable salt or N-oxide thereof, wherein
X is selected from the group consisting of N and CH;
Y is selected from the group consisting of O, S, SO, SO₂, SONR⁴¹, NH, and NR⁴²;
Z is selected from the group consisting of O, S, SO, SO₂, SONR⁴¹, NH, and NR⁴²;
each R⁴¹, independently of the others, is hydrogen or lower alkyl;
each R⁴², independently of the others, is selected from the group consisting of alkyl, substituted alkyl, alkenyl, substituted alkenyl, alkynyl, substituted alkynyl, cycloalkynyl, substituted cycloalkynyl, aryl, substituted aryl, cycloalkyl, substituted cycloalkyl, heteroaryl, substituted heteroaryl, heterocyclyl, substituted heterocyclyl, hydroxyl, alkoxy, substituted alkoxy, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxyl ester, (carboxyl ester)oxy, and —(CRR)ₙ—R^{p}, wherein
each R, independently of the others, is selected from the group consisting of hydrogen, lower alkyl and halo;
n is an integer from 0 to 4; and
R^{p} is selected from the group consisting of phosphate, phosphate ester, phosphonate, phosphorodiamidate, phosphoramidate monoester, phosphoramidate diester, cyclic phosphoramidate, cyclic phosphorodiamidate, phosphonamidate, and cyclic phosphonamidate;
each R⁴³, independently of the others, is selected from the group consisting of hydrogen, alkyl, substituted alkyl, alkoxy, substituted alkoxy, alkenyl, substituted alkenyl, alkynyl, substituted alkynyl, cycloalkynyl, substituted cycloalkynyl, alkynyloxy, amino, substituted amino, aryl, substituted aryl, aryloxy, substituted aryloxy, cyano, cycloalkyl, substituted cycloalkyl, cycloalkoxy, substituted cycloalkoxy, heteroaryl, substituted heteroaryl, heteroaryloxy, substituted heteroaryloxy, heterocyclyl, substituted heterocyclyl, heterocyclyloxy, substituted heterocyclyloxy, aminocarbonyl, aminocarbonyloxy, carboxyl, carboxyl ester, (carboxyl ester)oxy, nitro, and halo, or, alternatively, two R⁴³ bonded to the same carbon atom are taken together to form an oxo (═O), ═NH or ═NR⁴⁴ group and the other two R⁴³ are as defined above;
each R⁴⁴, independently of the others, is selected from the group consisting of (C1-C6) alkyl and (C5-C14) aryl; and
R⁴⁵ is selected from the group consisting of alkyl, substituted alkyl, alkoxy, substituted alkoxy, amino, substituted amino, carboxyl, carboxyl ester, cyano, halo, nitro, alkenyl, substituted alkenyl, alkynyl and substituted alkynyl.
